(19) United States
(12) Patent Application Publication
(10) Pub. No.: US 2008/0021557 A1
(43) Pub. Date: Jan. 24, 2008

(54) Spinal motion-preserving implants

(75) Inventor: Hai H. Trieu, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/491,783

(22) Filed: Jul. 24, 2006

(51) Int. Cl. A61F 2/44

(52) U.S. Cl. 623/17.15

(57) ABSTRACT

In a particular embodiment, a prosthetic device is provided which includes a component that includes a rigid-rod polymer material and is configured to be implanted in association with two vertebrae.

### FIELD OF THE DISCLOSURE

This disclosure, in general, relates to implantable devices and particularly to implantable devices for implantation in and around the spine.

### BACKGROUND

In human anatomy, the spine is a generally flexible column that can withstand tensile and compressive loads. The spine also allows bending motion and provides a place of attachment for keels, muscles, and ligaments. Generally, the spine is divided into four sections: the cervical spine, the thoracic or dorsal spine, the lumbar spine, and the pelvic spine. The pelvic spine generally includes the sacrum and the coccyx. The sections of the spine are made up of individual bones called vertebrae. Three joints reside between each set of two vertebrae: a larger intervertebral disc between the two vertebral bodies and two zygapophysial joints located posteriolaterally relative to the vertebral bodies and between opposing articular processes.

The intervertebral discs generally function as shock absorbers and as joints. Further, the intervertebral discs can absorb the compressive and tensile loads to which the spinal column can be subjected. At the same time, the intervertebral discs can allow adjacent vertebral bodies to move relative to each other, particularly during bending or flexure of the spine. Thus, the intervertebral discs are under constant muscular and gravitational stress and generally, the intervertebral discs are the first parts of the lumbar spine to show signs of deterioration.

The zygapophysial joints permit movement in the vertical direction, while limiting rotational motion of two adjoining vertebrae. In addition, capsular ligaments surround the zygapophysial joints, discouraging excess extension and torsion. In addition to intervertebral disc degradation, zygapophysial joint degeneration is also common because the zygapophysial joints are frequently in motion with the spine. In fact, zygapophysial joint degeneration and disc degeneration frequently occur together. Generally, although one can be the primary problem while the other is a secondary problem resulting from the altered mechanics of the spine, by the time surgical options are considered, both zygapophysial joint degeneration and disc degeneration typically have occurred.

Deterioration of the spine in general can be manifested in many different forms, including, spinal stenosis, degenerative spondylolisthesis, degenerative scoliosis, or a herniated disc, or sometimes a combination of these problems. Accordingly the industry continues to seek new ways to prevent and improve the condition of the spine in patients. Particularly, the medical industry seeks improved devices and procedures to combat the various maladies associated with the spine.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of a lateral view of a portion of a vertebral column.

FIG. 2 includes an illustration of a lateral view of a pair of adjacent vertebrae.

FIG. 3 includes an illustration of a top plan view of a vertebra.

FIG. 4 includes an illustration of a top view of an intervertebral disc.

FIG. 5 includes an illustration of a cross-sectional view of two adjacent vertebrae.

FIG. 6, FIG. 7, FIG. 8, FIG. 9, and FIG. 10 include illustrations of an exemplary embodiment of a prosthetic disc implant.

FIG. 11 and FIG. 12 include illustrations of an exemplary prosthetic disc implanted between two vertebrae.

FIG. 13, FIG. 14, FIG. 15, FIG. 16, FIG. 17, FIG. 18, FIG. 19, FIG. 20, and FIG. 21 include illustrations of exemplary embodiments of prosthetic disc implants.

FIG. 22, FIG. 23, FIG. 24, FIG. 25, FIG. 26, FIG. 27, FIG. 28, FIG. 29, and FIG. 30 include illustrations of exemplary embodiments of nucleus implantable devices.

FIG. 31 includes an illustration of an exemplary implantable device kit.

The use of the same reference symbols in different drawings indicates similar or identical items.

### DESCRIPTION OF THE DRAWINGS

In a particular embodiment, an implantable device includes a component that includes a rigid-rod polymer material and is configured to be implanted in association with two vertebrae. For example, the component can have a surface that is subject to frictional forces. The surface can be formed of the rigid-rod polymer. In another example, the component can have a contact surface that contacts an osteal structure. The contact surface can be formed of the rigid-rod polymer.

In a particular embodiment, a prosthetic device is provided which includes a component that includes a rigid-rod polymer material and is configured to be implanted in association with two vertebrae.

In another exemplary embodiment, an implantable device includes a component configured to be implanted in association with two vertebrae, the component including a polymeric material including a rigid-rod polymer matrix.

In another exemplary embodiment, an implantable device includes a first component configured to be implanted in association with two vertebrae, such that the first component has a first surface configured to moveable engage an opposing second surface, the first surface can include a rigid-rod polymer material. The device also includes a second component having the opposing second surface.

In a further exemplary embodiment, an implantable device includes a first component having a depression formed therein and a second component having a projection extending therefrom, such that the projection includes a surface configured to movably engage the depression. Additionally, at least one of the first component or the second component includes a rigid-rod polymer material, and device is configured to be installed between two vertebrae.

### Description of Relevant Anatomy

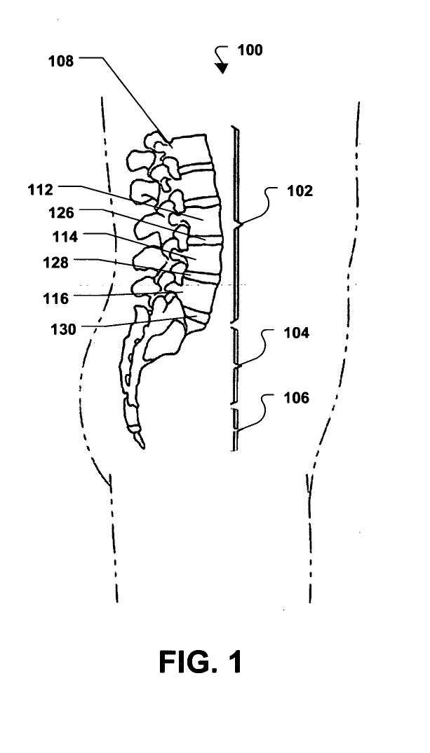

Referring initially to FIG. 1, a portion of a vertebral column, designated 100, is shown. As depicted, the vertebral column 100 includes a lumbar region 102, a sacral region 104, and a coccygeal region 106. The vertebral column 100 also includes a cervical region and a thoracic region. For clarity and ease of discussion, the cervical region and the thoracic region are not illustrated.

As illustrated in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, or damaged or if one of the zygapophysial joints is diseased, degenerated or damaged, that disc or joint can be at least partially treated with an implanted device according to one or more of the embodiments described herein. In a particular embodiment, a disc replacement device can be inserted into the intervertebral lumbar disc 122, 124, 126, 128, 130 or a zygapophysial joint.

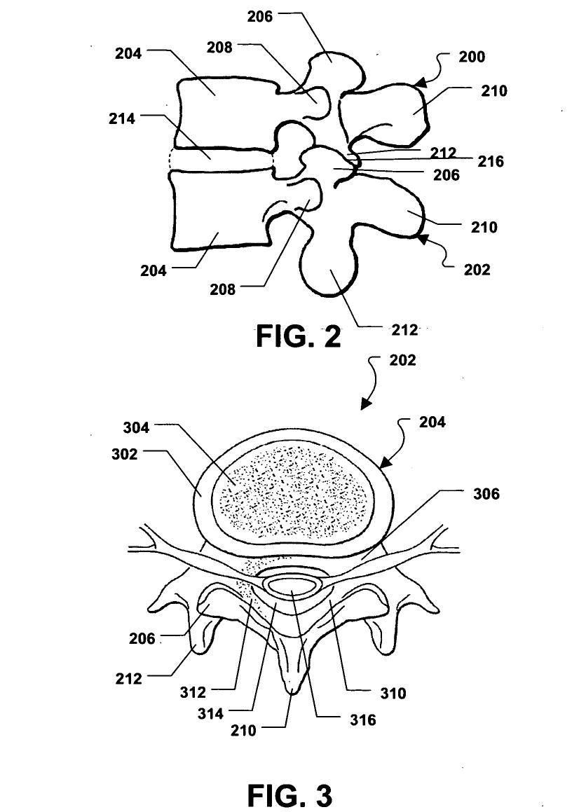

FIG. 2 depicts a detailed lateral view of two adjacent vertebrae, e.g., two of the lumbar vertebrae 108, 110, 112, 114, 116 illustrated in FIG. 1. FIG. 2 illustrates a superior vertebra 200 and an inferior vertebra 202. As illustrated, each vertebra 200, 202 includes a vertebral body 204, a superior articular process 206, a transverse process 208, a spinous process 210 and an inferior articular process 212. FIG. 2 further depicts an intervertebral disc 214 between the superior vertebra 200 and the inferior vertebra 202. A zygapophysial joint 216 is located between the inferior articular process 212 of the superior vertebra 200 and the superior articular process 206 of the inferior vertebra 202. As described in greater detail below, an implantable device according to one or more of the embodiments described herein can be installed within or in proximity to the intervertebral disc 214 between the superior vertebra 200 and the inferior vertebra 202 or within or in proximity to the zygapophysial joint 216.

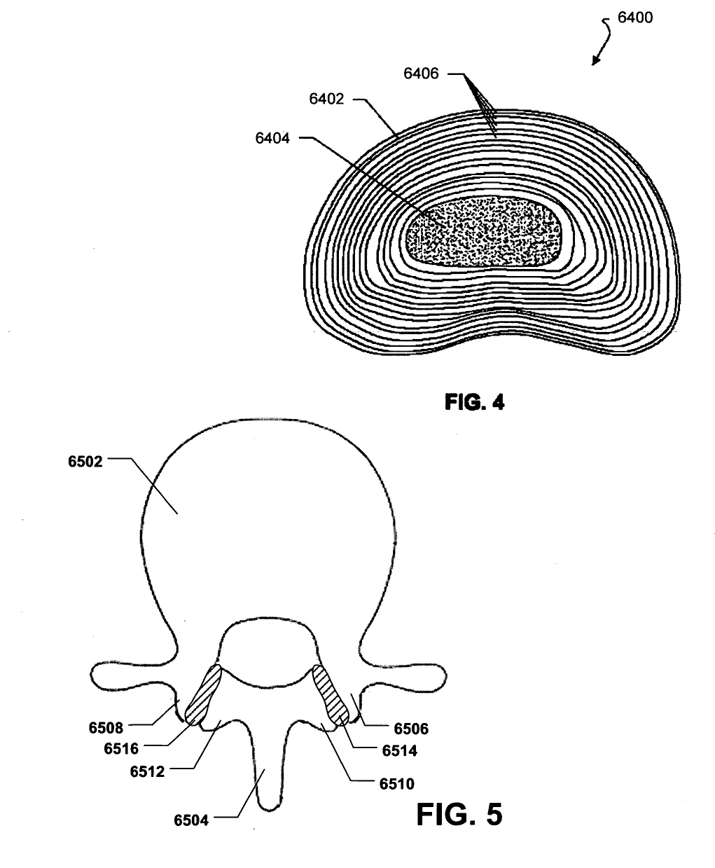

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is generally softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

Referring now to FIG. 4, an intervertebral disc is shown and is generally designated 6400. The intervertebral disc 6400 is made up of two components: an annulus fibrosis 6402 and a nucleus pulposus 6404. The annulus fibrosis 6402 is the outer portion of the intervertebral disc 6400, and the annulus fibrosis 6402 includes a plurality of lamellae 6406. The lamellae 6406 are layers of collagen and proteins. Each lamella 6406 typically includes fibers that slant at 30-degree angles, and the fibers of each lamella 6406 run in a direction opposite the adjacent layers. Accordingly, the annulus fibrosis 6402 is a structure that is exceptionally strong, yet extremely flexible.

The nucleus pulposus 6404 is an inner gel material that is surrounded by the annulus fibrosis 6402. It makes up about forty percent (40%) of the intervertebral disc 6400 by weight. Moreover, the nucleus pulposus 6404 can be considered a ball-like gel that is contained within the lamellae 6406. The nucleus pulposus 6404 includes loose collagen fibers, water, and proteins. The water content of the nucleus pulposus 6404 is about ninety percent (90%) by weight at birth and decreases to about seventy percent by weight (70%) by the fifth decade.

Injury or aging of the annulus fibrosis 6402 can allow the nucleus pulposus 6404 to be squeezed through the annulus fibers either partially, causing the disc to bulge, or completely, allowing the disc material to escape the intervertebral disc 6400. The bulging disc or nucleus material can compress the nerves or spinal cord, causing pain. Accordingly, the nucleus pulposus 6404 can be treated or replaced with an implantable device to improve the condition of the intervertebral disc 6400.

FIG. 5 includes a cross-sectional view of the spine illustrating a portion of a superior vertebra 6504 and a portion of an inferior vertebra 6502. The inferior vertebra 6502 includes superior articular processes 6506 and 6508 and the superior vertebra 6504 includes inferior articular processes 6510 and 6512. Between the superior articular process 6506 and the inferior articular process 6510 is a zygapophysial joint 6514 and between the superior articular process 6508 and the inferior articular process 6512 is a zygapophysial joint 6516.

When damaged or degraded, the zygapophysial joints 6514 and 6516 can be treated. For example, an implantable device can be inserted into or in proximity to the zygapophysial joints 6514 and 6516. In particular, such an implantable device can be configured to fit between the inferior articular process (6506 or 6508) and the superior articular process (6510 or 6512).

### Description of Materials for Use in Implantable Devices

In general, components of implantable devices are formed of biocompatible materials. For example, components can be formed of a metallic material, ceramic material, or of a polymeric material. An exemplary metallic material includes titanium, titanium alloy, tantalum, tantalum alloy, zirconium, zirconium alloy, stainless steel, cobalt, cobalt containing alloy, chromium containing alloy, indium tin oxide, silicon, magnesium containing alloy, aluminum, aluminum containing alloy, or any combination thereof.

Exemplary ceramic materials generally include oxides, carbides, or nitrides. More particularly, ceramics can include oxides, for example, aluminum oxide and zirconium oxide. An exemplary carbide includes titanium carbide. Ceramics can also generally include carbon containing compounds, including graphite, carbon fiber, or pyrolytic carbon to name a few examples.

The polymer materials of components of implantable devices are generally biocompatible. An exemplary polymeric material can include a polyurethane material, a polyolefin material, a polystyrene, a polyurea, a polyamide, a polyaryletherketone (PAEK) material, a silicone material, a hydrogel material, a rigid-rod polymer, or any alloy, blend or copolymer thereof. Particular polymers are also resorbable in vivo and a resorbable polymer can be gradually moved from the implantable device, either through degradation or solvent effects produced in vivo.

An exemplary polyolefin material can include polypropylene, polyethylene, halogenated polyolefin, flouropolyolefin, polybutadiene, or any combination thereof. An exemplary polyaryletherketone (PAEK) material can include polyetherketone (PEK), polyetheretherketone (PEEK), polyetherketoneketone (PEKK), polyetherketoneetherketoneketone (PEKEKK), or any combination thereof. An exemplary silicone can include dialkyl silicones, fluorosilicones, or any combination thereof. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof.

In a particular embodiment, a component of the device includes a rigid-rod polymer. In particular, the rigid-rod polymer can be a phenylene-based polymer, such as a homopolymer or a copolymer in which phenylene forms a portion of the polymeric chain in contrast to forming a functional group extending from the polymeric chain. Depending on the nature of copolymer monomers and functional groups, a rigid-rod polymer can form a crystalline phase that can provide strength or can provide conductivity.

Particular rigid-rod polymers can include copolymers that, in addition, to a phenylene group, include a benzoyl, an azole, a thiazole, an oxazol, a terephthalate group, or any combination thereof in the polymer chain. In a particular example, the rigid-rod polymer can include poly(phenylene benzobisthiazole) (PPBT), such as poly(p-phenylene benzobisthiazole). In another example, the rigid-rod polymer can include poly(phenylene benzobisoxazole) (PBO), such as poly(p-phenylene benzobisoxazole). In a further example, the rigid-rod polymer can include poly(phenylene benzimidazole) (PDIAB), such as poly(p-phenylene benzimidazole). In an additional example, the rigid-rod polymer can include poly(phenylene terephthalate) (PPTA), such as poly(p-phenylene terephthalate). In another example, the rigid-rod polymer can include poly(benzimidazole) (ABPBI), such as poly(2,5(6)benzimidazole). In a further example, the rigid-rod polymer can include poly(benzoyl-1,4-phenylene-co-1,3-phenylene). In addition, the rigid-rod polymer can include any combination of the above copolymers. A particular rigid-rod polymer can include a polymer sold under the trademark PARMAX®, available from Mississippi Polymer Technology, Inc. of Bay St. Louis, Miss.

In addition, a particular rigid-rod polymer can be thermoplastic. In another example, a particular rigid-rod polymer can be dissolved in solvent. Such a rigid-rod polymer can be formed into complex shapes.

Further, a particular rigid-rod polymer can have a high crystallinity. For example, the rigid-rod polymer can have a crystallinity of at least about 30%, such as at least about 50%, or even, at least about 65%. Alternatively, the rigid-rod polymer can be amorphous.

A component of an implantable device can be formed of a polymeric material. In a particular example, the polymeric material can include a rigid-rod polymer. For example, the polymeric material can consist essentially of the rigid-rod polymer. In another example, the rigid-rod polymer can form a rigid-rod polymer matrix surrounding a filler. In a further example, the polymeric material can include a polymer blend.

In a particular example, the polymeric material can be substantially rigid-rod polymer, such as consisting essentially of rigid-rod polymer. In particular, the polymeric material can be a thermoplastic rigid-rod polymer absent or substantially free of filler.

In another example, the polymeric material can include a rigid-rod polymer matrix surrounding a filler. The filler can be a particulate filler, a fiber filler, or any combination thereof. In an example, the filler can include a ceramic, a metal, a carbon, a polymer, or any combination thereof. For example, the filler can include a ceramic, such as a ceramic oxide, a boride, a nitride, a carbide, or any combination thereof. In another example, the filler can include a metal, such as a particulate metal or metal fiber. An exemplary metal can include titanium, titanium alloy, tantalum, tantalum alloy, zirconium, zirconium alloy, stainless steel, cobalt, cobalt containing alloy, chromium containing alloy, indium tin oxide, silicon, magnesium containing alloy, aluminum, aluminum containing alloy, or any combination thereof. In another exemplary embodiment, the filler can include a carbon, such as carbon black, diamond, graphite, or any combination thereof. For example, a rigid-rod polymer matrix can be reinforced with a carbon fiber. In a further exemplary embodiment, the filler can include a polymer, such as a polymer particulate or a polymer fiber. The polymer can be, for example, a polyurethane material, a polyolefin material, a polystyrene, a polyurea, a polyamide, a polyaryletherketone (PAEK) material, a silicone material, a hydrogel material, a rigid-rod polymer, or any alloy, blend or copolymer thereof. In an additional exemplary embodiment, the filler can include an agent, such as an agent absorbed in a carrier or a powdered agent.

In an exemplary embodiment, the polymeric material includes the rigid-rod polymer matrix and not greater than about 50 wt % of the filler. For example, the polymeric material can include not greater than about 30 wt % of the filler, such as not greater than about 15 wt % of the filler. Alternatively, the polymeric material can be self-reinforced and can be substantially free of the filler.

In another exemplary embodiment, the polymeric material can be a polymer blend. For example, the polymer blend can be a homogeneous polymer blend in which a rigid-rod polymer and at least one other polymer form a single phase. In another example, the polymer blend can be a heterogeneous polymer blend in which a rigid-rod polymer and at least one other polymer form separate, yet intertwined phases. In particular, the polymer blend can include at least about 25 wt % of the rigid-rod polymer, such as at least about 30 wt %, at least about 50 wt % of the rigid-rod polymer, or even, at least about 75 wt % of the rigid-rod polymer. The at least one other polymer can be selected from a polyurethane material, a polyolefin material, a polystyrene, a polyurea, a polyamide, a polyaryletherketone (PAEK) material, a silicone material, a hydrogel material, a rigid-rod polymer, or any alloy, blend or copolymer thereof. Whether the blend is homogeneous or heterogeneous can depend on the selection of the rigid-rod polymer and the at least one other polymer, in addition to processing parameters and techniques.

In a particular exemplary embodiment, the polymer blend can be a heterogeneous blend in which the rigid-rod polymer is blended with a resorbable polymer, such as polylactic acid (PLA) or the like. Once implanted, the resorbable polymer may degrade or migrate leaving a rigid-rod polymer matrix having osteoconductive properties.

In another exemplary embodiment, the polymer blend can include a rigid-rod polymer blended with a second polymer to alter the modulus of the rigid-rod polymer. In a further exemplary embodiment, the polymer blend can include an agent, such as osteogenerative agent, a stimulating agent, a degradation agent, an analgesic, an anesthetic agent, an antiseptic agent, or any combination thereof. For example, the polymer blend can include the rigid-rod polymer and a hydrogel. The hydrogel can include an agent.

The polymer material including a rigid-rod polymer can have desirable physical and mechanical properties. For example, the polymer material can have a glass transition temperature of at least about 145° C., such as at least about 155° C., based on ASTM E1356.

In an example, the polymeric material can have an ultimate tensile strength at room temperature (23° C.) of at least about 125 MPa, such as at least about 135 MPa, at least about 150 MPa, at least about 180 MPa, or even, at least about 200 MPa, based on ASTM D638. In addition, the polymer material can exhibit an average tensile modulus at room temperature (23° C.) of at least about 5.0 GPa. For example, the polymer material can exhibit a tensile modulus of at least about 6.0 GPa, such as at least about 7.5 GPa. Further, the polymer material can have an elongation of about 1% to about 5%, such as about 2% to about 4%.

In a further example, the polymeric material including a rigid-rod polymer can exhibit a flexural yield strength at room temperature of at least about 220 MPa, such as at least about 250 MPa, or even at least about 300 MPa, based on ASTM D790. In addition, the polymeric material can exhibit a flexural modulus at room temperature (23° C.) of at least about 5.0 GPa, such as at least about 6.0 GPa, or even, at least about 7.5 GPa. Further, the polymeric material can exhibit a compressive yield strength at room temperature (23° C.) of at least about 230 MPa, such as at least about 300 MPa, or even, at least about 400 MPa, based on ASTM D695.

For a particular rigid-rod polymer, the mechanical properties of the polymeric material can be direction dependent. Alternatively, a particular rigid-rod polymer can provide a polymeric material having near isotropic mechanical properties, such as substantially isotropic mechanical properties.

Despite the strength of polymeric material including rigid-rod polymer, the polymeric material can have a low specific gravity. For example, the polymeric material can have a specific gravity not greater than about 1.5, such as not greater than about 1.4, or even, not greater than about 1.3. Particular polymeric materials formed of a rigid-rod polymer can have a specific gravity not greater than about 1.26, such as not greater than about 1.23, or even not greater than about 1.21, based on ASTM D792.

Further particular polymeric materials including rigid-rod polymer can exhibit low water absorption, such as a water hydration of not greater than 1.0% at equilibrium, based on ASTM D570. For example, the polymeric material can exhibit a water hydration not greater than about 0.7%, such as not greater than about 0.55%.

In a further example, polymeric materials including a rigid-rod polymer can form smooth surfaces, such as polished surfaces having low roughness (Ra). For example, the polymer material can form a surface having a roughness (Ra) not greater than about 100 nm. Particular polymeric materials including a rigid-rod polymer can form a surface having a roughness (Ra) not greater than about 10 nm, such as not greater than about 1.0 nm. In particular, a polymeric material formed of a rigid-rod polymer absent a filler can form a smooth surface. Such surfaces, can be used to form wear resistant surfaces that are subject to movement against an opposing surface, such as opposing surfaces of an intervertebral disc replacement. In another example, a polymeric material including a rigid-rod polymer in a polymer blend can form a smooth surface. Alternatively, the polymeric material can be roughened, shaped, or convoluted to form a rough surface. Such surfaces are particularly suited for engaging osteal structures, such as vertebrae.

In an additional embodiment, the polymeric material including a rigid-rod polymer can coat a metallic article. For example, a rigid-rod polymer can coat a titanium component. In a particular example, a polymeric material including a rigid-rod polymer can be molded over a metallic component. Alternatively, the polymeric material including a rigid-rod polymer can be laminated to the metallic component, adhered to the metallic component, or mechanically fastened to the metallic component.

### Description of Agents

In an exemplary embodiment, an implantable device can include at least one reservoir, coating, or impregnated material configured to release an agent. The agent can generally affect a condition of proximate soft tissue, such as ligaments, a nucleus pulposus, an annulus fibrosis, or a zygapophysial joint, or can generally affect bone growth. For example, the agent can decrease the hydration level of the nucleus pulposus or can cause a degeneration of soft tissue, such as the nucleus pulposus, that leads to a reduction in hydration level, to a reduction in pressure, or to a reduction in size of, for example, the nucleus pulposus within the intervertebral disc. An agent causing a degeneration of soft tissue or a reduction in hydration level is herein termed a “degradation agent.” In another example, an agent can increase the hydration level of soft tissue, such as the nucleus pulposus, or can cause a regeneration of the soft tissue that results in an increase in hydration level or in an increase in pressure within the intervertebral disc, for example. Such an agent that can cause an increase in hydration or that can cause a regeneration of the soft tissue is herein termed a “regenerating agent.” In a further example, an agent (herein termed a “therapeutic agent”) can inhibit degradation of soft tissue or enhance maintenance of the soft tissue. Herein, therapeutic agents and regenerating agents are collectively referred to as “stimulating agents.” In a further example, an agent (e.g., an osteogenerative agent) can affect bone growth in proximity to the intervertebral disc or the zygapophysial joint. For example, an osteogenerative agent can be an osteoinductive agent, an osteoconductive agent, or any combination thereof.

An exemplary degradation agent can reduce hydration levels in the nucleus pulposus or can degrade the soft tissue, resulting in a reduction in hydration level or in pressure within the intervertebral disc, for example. For example, the degradation agent can be a nucleolytic agent that acts on portions of a nucleus pulposus. In an example, the nucleolytic agent is proteolytic, breaking down proteins.

An exemplary nucleolytic agent includes a chemonucleolysis agent, such as chymopapain, collagenase, chondroitinase, keratanase, human proteolytic enzymes, papaya protenase, or any combination thereof. An exemplary chondroitinase can include chondroitinase ABC, chondroitinase AC, chondroitinase ACII, chondroitinase ACIII, chondroitinase B, chondroitinase C, or the like, or any combination thereof. In another example, a keratanase can include endo-β-galactosidase derived from Escherichia freundii, endo-β-galactosidase derived from Pseudomonas sp. IFO-13309 strain, endo-β-galactosidase produced by Pseudomonas reptilivora, endo-β-N-acetylglucosaminidase derived from Bacillus sp. Ks36, endo-β-N-acetylglucosaminidase derived from Bacillus circulans KsT202, or the like, or any combination thereof. In a particular example, the degradation agent includes chymopapain. In another example, the degradation agent includes chondroitinase-ABC.

An exemplary regenerating agent includes a growth factor. The growth factor can be generally suited to promote the formation of tissues, especially of the type(s) naturally occurring as components of an intervertebral disc or of a zygapophysial joint. For example, the growth factor can promote the growth or viability of tissue or cell types occurring in the nucleus pulposus, such as nucleus pulposus cells or chondrocytes, as well as space filling cells, such as fibroblasts, or connective tissue cells, such as ligament or tendon cells. Alternatively or in addition, the growth factor can promote the growth or viability of tissue types occurring in the annulus fibrosis, as well as space filling cells, such as fibroblasts, or connective tissue cells, such as ligament or tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

An exemplary therapeutic agent can include a soluble tumor necrosis factor α-receptor, a pegylated soluble tumor necrosis factor α-receptor, a monoclonal antibody, a polyclonal antibody, an antibody fragment, a COX-2 inhibitor, a metalloprotease inhibitor, a glutamate antagonist, a glial cell derived neurotrophic factor, a B2 receptor antagonist, a substance P receptor (NK1) antagonist, a downstream regulatory element antagonistic modulator (DREAM), iNOS, an inhibitor of tetrodotoxin (TTX)-resistant Na+-channel receptor subtypes PN3 and SNS2, an inhibitor of interleukin, a TNF binding protein, a dominant-negative TNF variant, Nanobodies™, a kinase inhibitor, or any combination thereof. Another exemplary therapeutic agent can include Adalimumab, Infliximab, Etanercept, Pegsunercept (PEG sTNF-R1), Onercept, Kineret®, sTNF-R1, CDP-870, CDP-571, CNI-1493, RDP58, ISIS 104838, 1→3-β-D-glucan, Lenercept, PEG-sTNFRII Fc Mutein, D2E7, Afelimomab, AMG 108, 6-methoxy-2-napthylacetic acid or betamethasone, capsaiein, civanide, TNFRc, ISIS2302 and GI 129471, integrin antagonist, alpha-4 beta-7 integrin antagonist, cell adhesion inhibitor, interferon gamma antagonist, CTLA4-Ig agonist/antagonist (BMS-188667), CD40 ligand antagonist, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-1 0, HuMax IL-15 (anti-IL 15 antibody), or any combination thereof.

An osteogenerative agent, for example, can encourage the formation of new bone (“osteogenesis”), such as through inducing bone growth (“osteoinductivity”) or by providing a structure onto which bone can grow (“osteoconductivity”). Generally, osteoconductivity refers to structures supporting the attachment of new osteoblasts and osteoprogenitor cells. As such, the agent can form an interconnected structure through which new cells can migrate and new vessels can form. Osteoinductivity typically refers to the ability of the implantable device or a surface or a portion thereof to induce nondifferentiated stem cells or osteoprogenitor cells to differentiate into osteoblasts.

In an example, an osteoconductive agent can provide a favorable scaffolding for vascular ingress, cellular infiltration and attachment, cartilage formation, calcified tissue deposition, or any combination thereof. An exemplary osteoconductive agent includes collagen; a calcium phosphate, such as hydroxyapatite, tricalcium phosphate, or fluorapatite; demineralized bone matrix; or any combination thereof.

In another example, an osteoinductive agent can include bone morphogenetic proteins (BMP, e.g., rhBMP-2); demineralized bone matrix; transforming growth factors (TGF, e.g., TGF-β); osteoblast cells, growth and differentiation factor (GDF), LIM mineralized protein (LMP), platelet derived growth factor (PDGF), insulin-like growth factor (ILGF), or any combination thereof. In a further example, an osteoinductive agent can include HMG-CoA reductase inhibitors, such as a member of the statin family, such as lovastatin, simvastatin, pravastatin, fluvastatin, atorvastatin, cerivastatin, mevastatin, pharmaceutically acceptable salts esters or lactones thereof, or any combination thereof. With regard to lovastatin, the substance can be either the acid form or the lactone form or a combination of both. In a particular example, the osteoinductive agent includes a growth factor. In addition, osteoconductive and osteoinductive properties can be provided by bone marrow, blood plasma, or morselized bone of the patient, or other commercially available materials.

In addition, other agents can be incorporated into a reservoir, such as an antibiotic, an analgesic, an anti-inflammatory agent, an anesthetic, a radiographic agent, or any combination thereof. For example, a pain medication can be incorporated within a reservoir or a release material in which another agent is included or can be incorporated in a separate reservoir or release material. An exemplary pain medication includes codeine, propoxyphene, hydrocodone, oxycodone, or any combination thereof. In a further example, an antiseptic agent can be incorporated within a reservoir. For example, the antiseptic agent can include an antibiotic agent. In an additional example, a radiographic agent can be incorporated into a reservoir, such as an agent responsive to x-rays.

Each of the agents or a combination of agents can be maintained in liquid, gel, paste, slurry, solid form, or any combination thereof. Solid forms include powder, granules, microspheres, miniature rods, or embedded in a matrix or binder material, or any combination thereof. In an example, fluids or water from surrounding tissues can be absorbed by the device and placed in contact with an agent in solid form prior to release. Further, a stabilizer or a preservative can be included with the agent to prolong activity of the agent.

In particular, one or more agents can be incorporated into a polymeric matrix, such as a hydrogel, a bioresorbable polymer, or a natural polymer. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof. An exemplary bioresorbable polymer can include polylactide (PLA), polyglycolide (PGA), poly(lactide-co-glycolide) (PLGA), polyanhydride, polyorthoester, or any combination thereof. An exemplary natural polymer can include a polysaccharide, collagen, silk, elastin, keratin, albumin, fibrin, or any combination thereof.

### Embodiments of Implantable Device

According to an aspect, the implantable device includes a component configured to be implanted in association with two vertebrae. The component can include a polymeric material including a rigid-rod polymer. In general, the implantable devices provided herein can be implanted proximate to the spinal column, such as near or around the spinal column and more particularly, fixably attached to the spinal column. For clarity, the terms “spinal column” or “spine” as used herein, refers to all portions of the spine, including the bones, discs, muscles, and ligaments unless otherwise stated. Moreover, the components provided herein include articulating components that can engage the spine and preserve a certain degree of movement.

According to an embodiment, the component can include a first surface configured to movably engage an opposing second surface. According to another embodiment, the component includes a first surface that is configured to engage a second opposing surface such that the surfaces are configured to movably engage one another. Accordingly, the second opposing surface can be part of a second component and as such, the first and second components can be configured to articulate relative to each other. In an embodiment, the first and second components can be configured to engage at least one vertebrae and facilitate relative motion between a first vertebra and a second vertebra. In a particular embodiment, the first and second components can be configured to be installed between a first and second vertebrae, in an intervertebral disc space.

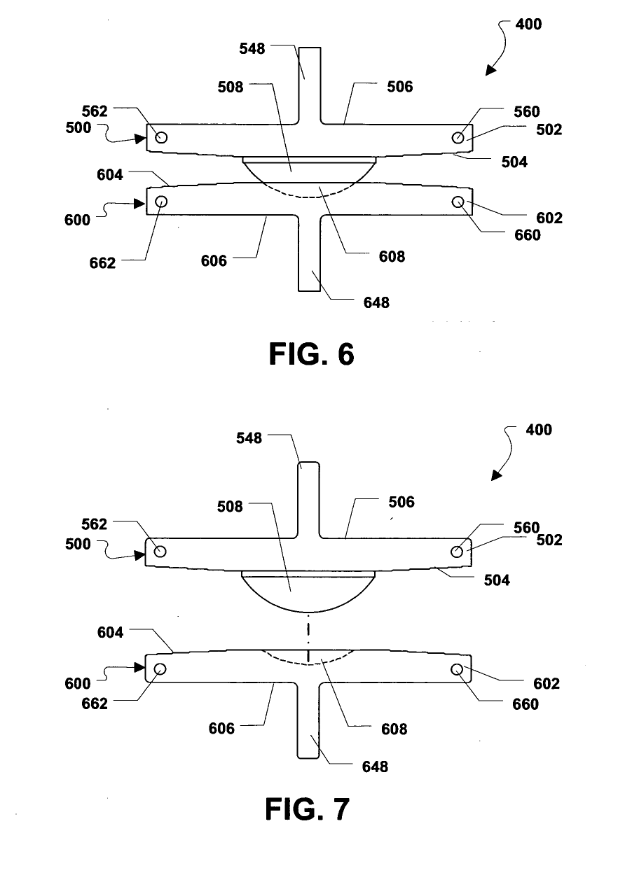

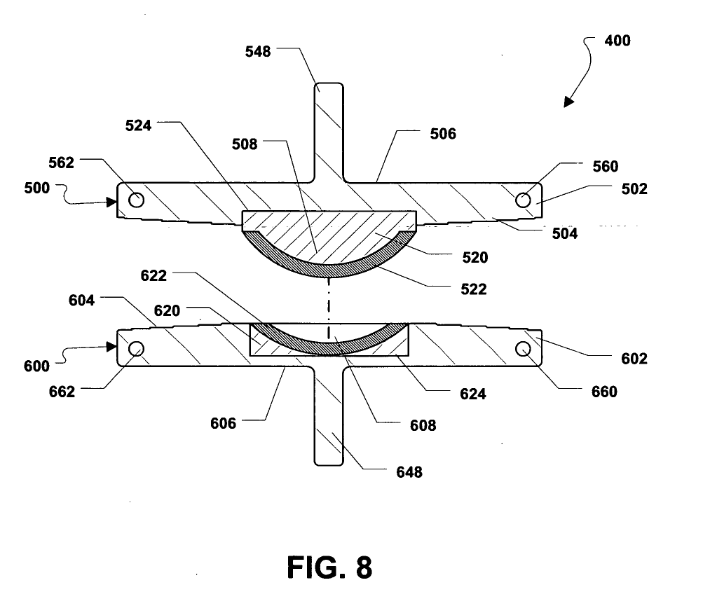

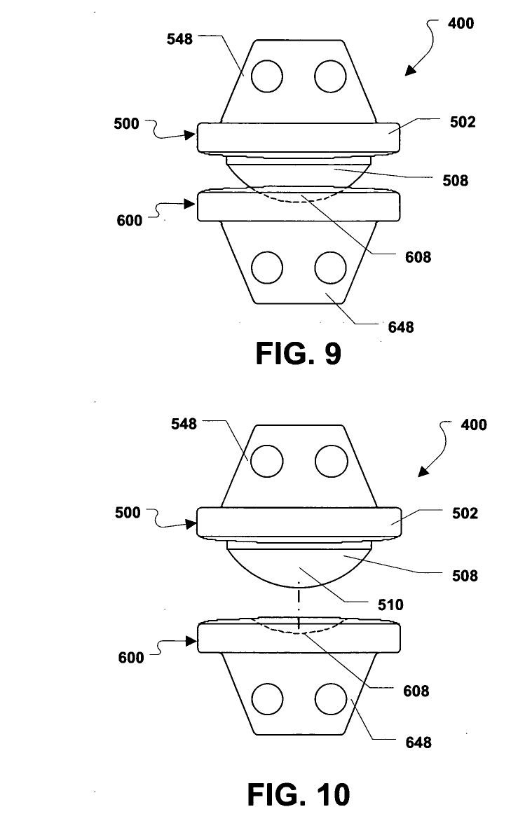

Referring to FIGS. 6 through 10, a first embodiment of an intervertebral prosthetic disc is shown and is generally designated 400. As illustrated, the intervertebral prosthetic disc 400 can include a superior component 500 and an inferior component 600. In a particular embodiment, the components 500, 600 can be made from one or more biocompatible materials. For example, the materials can be metal containing materials, polymer materials, or combinations thereof. The metal containing materials can be pure metals, metal alloys, or a metal containing a polymer or ceramic filler. The pure metals can include titanium. Moreover, the metal alloys can include stainless steel, a cobalt-chrome-molybdenum alloy, e.g., ASTM F-999 or ASTM F-75, a titanium alloy, or a combination thereof

In a particular embodiment, the components can include a polymer material, such as a polymeric material including a rigid-rod polymer. In a particular embodiment, the components can be formed essentially of a rigid-rod polymer material, such as a rigid-rod polymer material that is substantially free of fillers.

In a particular embodiment, the superior component 500 can include a superior support plate 502 that has a superior articular surface 504 and a superior bearing surface 506. In a particular embodiment, the superior articular surface 504 can be generally curved and the superior bearing surface 506 can be substantially flat. In an alternative embodiment, the superior articular surface 504 can be substantially flat and at least a portion of the superior bearing surface 506 can be generally curved.

As illustrated in FIG. 6 through FIG. 10, a projection 508 extends from the superior articular surface 504 of the superior support plate 502. In a particular embodiment, the projection 508 can have a hemi-spherical shape. Alternatively, the projection 508 can have an elliptical shape, a cylindrical shape, or another arcuate shape.

In a further embodiment illustrated in FIG. 8, the projection 508 can include a base 520 and a superior wear resistant layer 522 affixed to, deposited on, or otherwise disposed on, the base 520. In a particular embodiment, the base 520 can act as a substrate and the superior wear resistant layer 522 can be deposited on the base 520. Further, the base 520 can engage a cavity 524 that can be formed in the superior support plate 502. In a particular embodiment, the cavity 524 can be sized and shaped to receive the base 520 of the projection 508. Further, the base 520 of the projection 508 can be press fit into the cavity 524.

In a particular embodiment, the base 520 of the projection 508 can be formed of a metallic material, polymeric material, or combination thereof. In particular, the base 520 can be formed of a polymer, such as an elastomeric polymer, or more particularly a rigid rod polymer. In another example, the polymeric material forming the base 520 can include a filler, such as a ceramic filler or an inorganic, carbon-based substance, such as graphite. According to one embodiment, the base 520, and likewise, all portions of the superior component 500 can include a rigid-rod polymer material, such as a molded or formed rigid-rod polymer material. In one particular embodiment, the superior component 500 can be formed of a rigid-rod polymer material that is essentially free of any filler materials.

Further, in an exemplary embodiment, the superior wear resistant layer 522 can include polymeric material including a rigid-rod polymer that is deposited on the base 520. In a particular embodiment, the superior wear resistant layer 522 can be formed essentially of a rigid-rod polymer material having substantially no fillers. In an embodiment, the rigid-rod polymer material can be molded and formed to fit the contour of the base 520 and affixed using conventional bonding, fastening, forming or deposition techniques. Alternatively, the superior wear resistant layer can be co-molded with the base 520.

Accordingly, the base 520 can be made from a material that can bond to the rigid-rod polymer material. The base 520 can be fitted into a superior support plate 502 made from one or more of the materials described herein. Also, in a particular embodiment, the base 520 can be roughened prior to the placement of the superior wear resistant layer 522. For example, the base 520 can be roughened using a roughening process. In particular, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 520 on which the superior wear resistant layer 522 is placed can be serrated and can include one or more teeth, spikes, or other protrusions extending therefrom. The serrations of the base 520 can facilitate anchoring of the superior wear resistant layer 522 on the base 520 and can substantially reduce the likelihood of delamination of the superior wear resistant layer 522 from the base 520.

In a particular embodiment, the superior wear resistant layer 522 can have a thickness in a range of fifty micrometers to five millimeters (50 μm-5 mm). Further, the superior wear resistant layer 522 can have a thickness in a range of two hundred micrometers to two millimeters (200 μm-2 mm). In a particular embodiment, the serrations that can be formed on the surface of the base 520 can have a height that is at most half of the thickness of the superior wear resistant layer 522. Accordingly, the likelihood that the serrations will protrude through the superior wear resistant layer 522 is substantially minimized.

Additionally, in a particular embodiment, a Young's modulus of the superior wear resistant layer 522 can be substantially greater than a Young's modulus of the base 520. Also, a hardness of the superior wear resistant layer 522 can be substantially greater than a hardness of the base 520. Further, the superior wear resistant layer 522 can include a material having a substantially greater toughness than the material of the base 520. Also, the superior wear resistant layer 522 can be polished in order to minimize surface irregularities of the superior wear resistant layer 522 and increase a smoothness of the superior wear resistant layer 522.

As provided above, certain materials are well-suited to handle the mechanical requirements of the superior wear resistant layer 522. According to one particular embodiment, the superior wear resistant layer 522 can be made essentially of a rigid-rod polymer matrix and can be essentially free of a filler material. In another example, the superior wear resistant layer 522 can be formed of a polymer blend including rigid-rod polymer, such as a homogeneous polymer blend. In particular embodiments, use of a homogeneous rigid-rod polymer materials can provide a suitable surface roughness in combination with other desirable mechanical properties. In an embodiment, the surface roughness of the wear resistant layer 522 is not greater than about 100 nm, such as not greater than about 50 nm, or even not greater than about 10 nm. Still, in another embodiment, the surface roughness of the superior wear resistant layer 522 is not greater than about 1.0 nm.

FIG. 6 through FIG. 10 indicate that the superior component 500 can include a superior keel 548 that extends from superior bearing surface 506. During installation, described below, the superior keel 548 can at least partially engage a keel groove that can be established within a cortical rim of a vertebra. Further, the superior keel 548 can be coated with a bioactive agent such as an osteogenerative agent, e.g., a hydroxyapatite coating formed of calcium phosphate. Additionally, the superior bearing surface 506 can be roughened prior to being coated with the bone-growth promoting substance to further enhance bone growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Additionally, the superior keel 548 or the superior bearing surface 506, can be porous structures, having a porosity within a range of between about 10-50 vol %. Such porosity can facilitate delivery of an osteogenerative agent to the surrounding tissue and bone.

FIG. 6 through FIG. 8 show that the superior component 500 can include a first implant inserter engagement hole 560 and a second implant inserter engagement hole 562. In a particular embodiment, the implant inserter engagement holes 560, 562 are configured to receive respective dowels, or pins, that extend from an implant inserter (not shown) that can be used to facilitate the proper installation of an intervertebral prosthetic disc, e.g., the intervertebral prosthetic disc 400 shown in FIG. 6 through FIG. 10.

In a particular embodiment, the inferior component 600 can include an inferior support plate 602 that has an inferior articular surface 604 and an inferior bearing surface 606. In a particular embodiment, the inferior articular surface 604 can be generally curved and the inferior bearing surface 606 can be substantially flat. In an alternative embodiment, the inferior articular surface 604 can be substantially flat and at least a portion of the inferior bearing surface 606 can be generally curved.

As illustrated in FIG. 4 through FIG. 8, a depression 608 extends into the inferior articular surface 604 of the inferior support plate 602. In a particular embodiment, the depression 608 is sized and shaped to receive the projection 508 of the superior component 500. For example, the depression 608 can have a hemi-spherical shape. Alternatively, the depression 608 can have an elliptical shape, a cylindrical shape, or another arcuate shape.

Referring to an embodiment illustrated in FIG. 8, the depression 608 can include a base 620 and an inferior wear resistant layer 622 affixed to, deposited on, or otherwise disposed on, the base 620. In a particular embodiment, the base 620 can act as a substrate and the inferior wear resistant layer 622 can be deposited on the base 620. Further, the base 620 can engage a cavity 624 that can be formed in the inferior support plate 602. In a particular embodiment, the cavity 624 can be sized and shaped to receive the base 620 of the depression 608. Further, the base 620 of the depression 608 can be press fit into the cavity 624.

In a particular embodiment, the base 620 of the depression 608 can include a polymeric material including a rigid-rod polymer, such as a polymeric material consisting essentially of a rigid-rod polymer material and being essentially free of fillers. As with the superior wear resistant layer 522, the inferior wear resistant layer 622 can be formed from the same or substantially similar material and be formed on the surface of the base 620 in the same or substantially similar manner.

Also, in a particular embodiment, the base 620 can be roughened prior to the deposition of the inferior wear resistant layer 622 thereon. For example, the base 620 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 620 on which the inferior wear resistant layer 622 is placed can be serrated and can include one or more teeth, spikes, or other protrusions extending therefrom. The serrations of the base 620 can facilitate anchoring of the inferior wear resistant layer 622 on the base 620 and can substantially reduce the likelihood of delamination of layer 622 from the base 620.

In a particular embodiment, the inferior wear resistant layer 622 can have a thickness in a range of fifty micrometers to five millimeters (50 μm-5 mm). Further, the inferior wear resistant layer 622 can have a thickness in a range of two hundred micrometers to two millimeters (200 μm-2 mm). In a particular embodiment, the serrations that can be formed on the surface of the base 620 can have a height that is at most half of the thickness of the inferior wear resistant layer 622. Accordingly, the likelihood that the serrations will protrude through the inferior wear resistant layer 622 is substantially minimized.

Additionally, in a particular embodiment, a Young's modulus of the inferior wear resistant layer 622 can be substantially greater than a Young's modulus of the base 620. Also, a hardness of the inferior wear resistant layer 622 can be substantially greater than a hardness of the base 620. Further, a toughness of the inferior wear resistant layer 622 can be substantially greater than a toughness of the base 620. In a particular embodiment, the inferior wear resistant layer 622 can be annealed immediately after deposition in order to minimize cracking of the inferior wear resistant layer. Also, the inferior wear resistant layer 622 can be polished in order to minimize surface irregularities of the inferior wear resistant layer 622 and increase a smoothness of the inferior wear resistant layer 622.

As provided above in conjunction with the superior wear resistant layer 522, certain materials are well-suited to handle the mechanical requirements of the inferior wear resistant layer 622. According to one particular embodiment, the inferior wear resistant layer 622 can be formed essentially of a rigid-rod polymer matrix and can be essentially free of a filler material. In another example, the inferior wear resistant layer 622 can be formed of a polymer blend including rigid-rod polymer, such as a homogeneous polymer blend. In particular embodiments, use of homogeneous rigid-rod polymer materials can provide a suitable surface roughness in combination with other desirable mechanical properties. In an embodiment, the surface roughness of the wear resistant layer 622 is not greater than about 100 nm, such as not greater than about 50 nm, or even not greater than about 10 nm. Still, in another embodiment, the surface roughness of the inferior wear resistant layer 622 is not greater than about 1.0 nm.

FIG. 6 through FIG. 10 indicate that the inferior component 600 can include an inferior keel 648 that extends from inferior bearing surface 606. During installation, described below, the inferior keel 648 can at least partially engage a keel groove that can be established within a cortical rim of a vertebra. Further, the inferior keel 648 can be coated with an osteogenerative agent, e.g., a hydroxyapatite coating formed of calcium phosphate. Additionally, the inferior bearing surface 606 can be roughened prior to being coated with the bone-growth promoting substance to further enhance bone growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Additionally, the inferior keel 648 or the inferior bearing surface 606, can be porous structures, having a porosity within a range of between about 10-50 vol %. Such porosity can facilitate delivery of an osteogenerative agent to the surrounding tissue and bone.

FIG. 6 through FIG. 8 show that the inferior component 600 can include a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 400 shown in FIG. 6 through FIG. 10.

In a particular embodiment, the overall height of the intervertebral prosthetic device 400 can be in a range from fourteen millimeters to forty-six millimeters (14-46 mm). Further, the installed height of the intervertebral prosthetic device 400 can be in a range from eight millimeters to sixteen millimeters (8-16 mm). In a particular embodiment, the installed height can be substantially equivalent to the distance between an inferior vertebra and a superior vertebra when the intervertebral prosthetic device 400 is installed there between.

In a particular embodiment, the length of the intervertebral prosthetic device 400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 548, 648 can have a height in a range from three millimeters to fifteen millimeters (3-15 mm).

While the superior component 500 is illustrated in FIG. 8 as including multiple parts, the superior component 500 can be alternatively an integral part formed from a single material or formed from co-molded materials. Similarly, the inferior component 600 can be formed as an integral part formed from a single material or formed from co-molded materials. It will be appreciated that in addition to the wear resistant layers provided herein, other components, such as, for example, the base components, can include a rigid-rod polymer material. In fact, according to one embodiment, the superior component and inferior component can be single component, molded pieces, comprising essentially a rigid-rod polymer material.

It will also be appreciated that any of the wear resistant layers provided herein can include a rigid-rod polymer material that is suitable for articulating against another wear resistant layer of material including a metal, other polymer or ceramic. According to an embodiment, a wear resistant layer including a rigid-rod polymer material is configured to articulate against an adjacent wear resistant layer including a metal, such as titanium, titanium carbide, cobalt-chromium alloy, metal alloys thereof, or other metal alloys. In another embodiment, a wear resistant layer including a rigid-rod polymer material is configured to articulate against an adjacent wear resistant layer including another polymer material, such as PEEK, PEK, PEKK, UHMWPE, or the like. Still, according to another embodiment, a wear resistant layer including a rigid-rod polymer material is configured to articulate against an adjacent wear resistant layer including a ceramic, such as oxides, nitrides, carbides, other carbon-containing compounds, or the like. In a further embodiment, a wear resistant layer including a rigid-rod polymer material is configured to articulate against bone cartilage or soft tissue.

### Installation of the First Embodiment within an Intervertebral Space

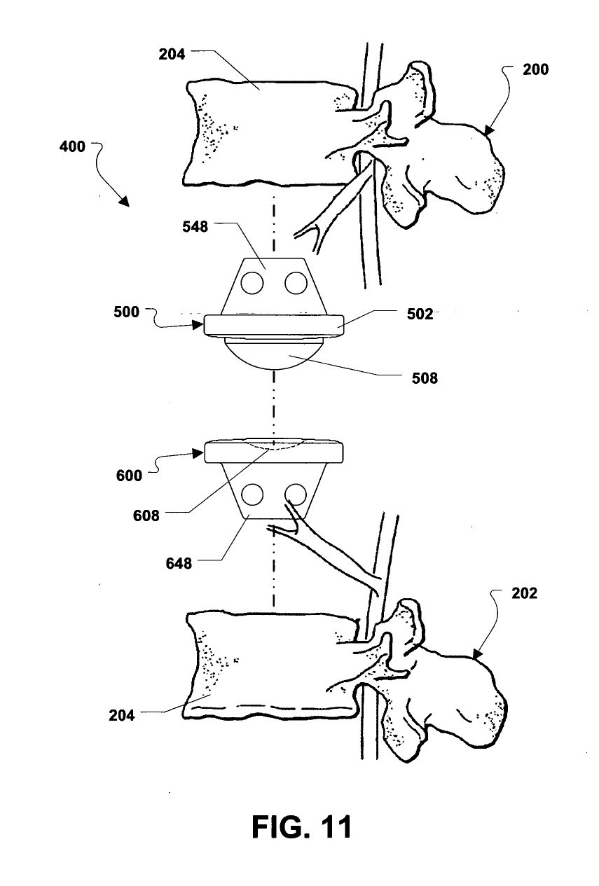

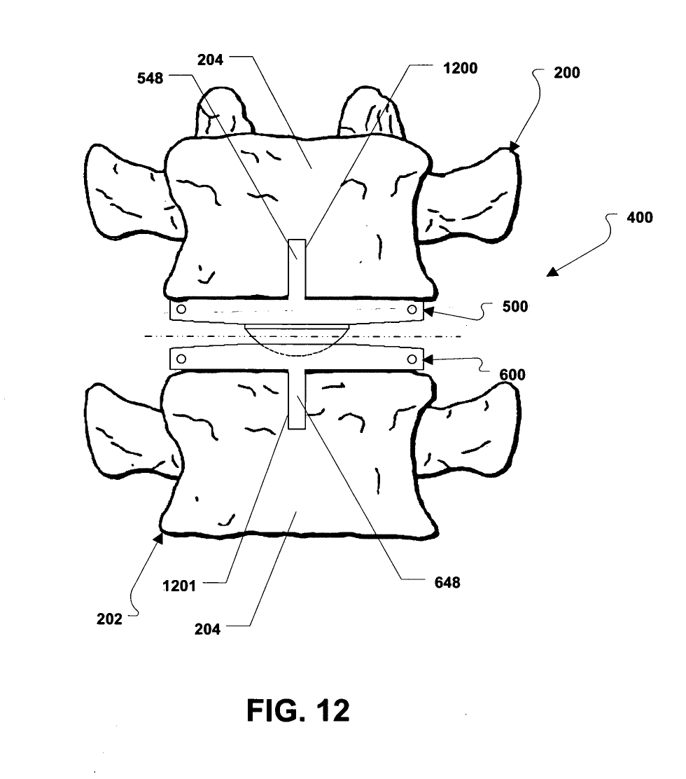

Referring to FIG. 11 and FIG. 12, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 6 through FIG. 10. Alternatively, the intervertebral prosthetic disc can be an intervertebral prosthetic disc according to any of the embodiments disclosed herein.

As shown in FIG. 11 and FIG. 12, the intervertebral prosthetic disc 400 can be installed within the intervertebral space 214 that can be established between the superior vertebra 200 and the inferior vertebra 202 by removing vertebral disc material (not shown). FIG. 12 shows that the superior keel 548 of the superior component 500 can at least partially engage the cancellous bone and cortical rim of the superior vertebra 200. Further, as shown in FIG. 12, the superior keel 548 of the superior component 500 can at least partially engage a superior keel groove 1200 that can be established within the vertebral body 204 of the superior vertebra 202. In a particular embodiment, the vertebral body 204 can be further cut to allow the superior support plate 502 of the superior component 500 to be at least partially recessed into the vertebral body 204 of the superior vertebra 200.

Also, as shown in FIG. 11, the inferior keel 648 of the inferior component 600 can at least partially engage the cancellous bone and cortical rim of the inferior vertebra 202. Further, as shown in FIG. 12, the inferior keel 648 of the inferior component 600 can at least partially engage the inferior keel groove 1201, which can be established within the vertebral body 204 of the inferior vertebra 202. In a particular embodiment, the vertebral body 204 can be further cut to allow the inferior support plate 602 of the inferior component 600 to be at least partially recessed into the vertebral body 204 of the inferior vertebra 200.

As illustrated in FIG. 11 and FIG. 12, the projection 508 that extends from the superior component 500 of the intervertebral prosthetic disc 400 can at least partially engage the depression 608 that is formed within the inferior component 600 of the intervertebral prosthetic disc 400. More specifically, the superior wear resistant layer 522 of the superior component 500 can at least partially engage the inferior wear resistant layer 622 of the inferior component 600. Further, the superior wear resistant layer 522 of the superior component 500 can movably engage the inferior wear resistant layer 622 of the inferior component 600 to allow relative motion between the superior component 500 and the inferior component 600.

It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202. In a particular embodiment, the intervertebral prosthetic disc 400 can allow angular movement in any radial direction relative to the intervertebral prosthetic disc 400.

Further, as depicted in FIGS. 11 and 12, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

### Description of a Second Embodiment of an Intervertebral Prosthetic Disc

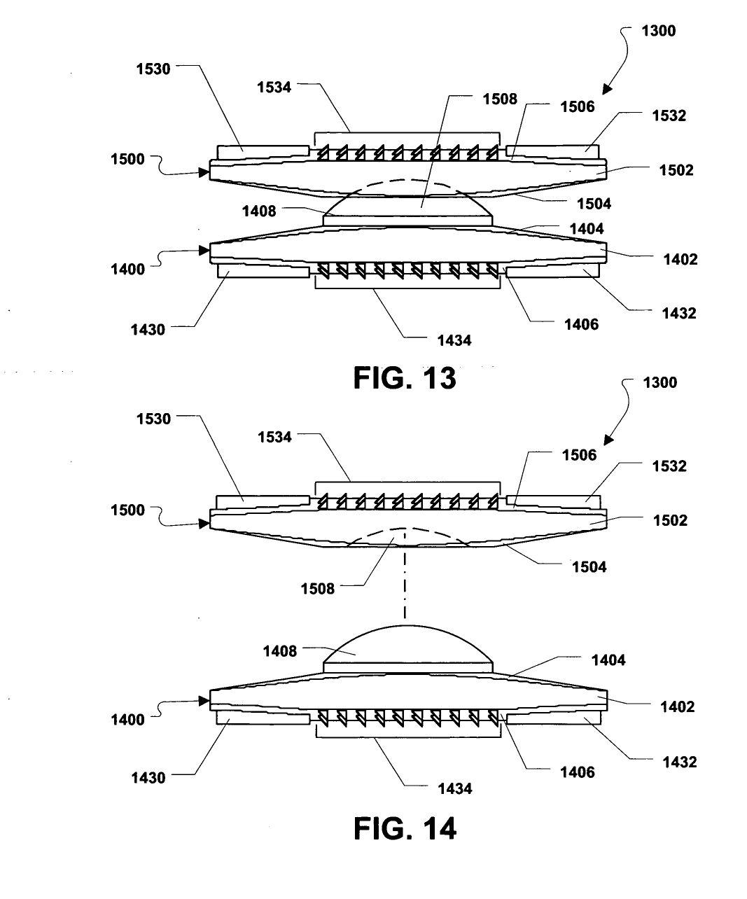

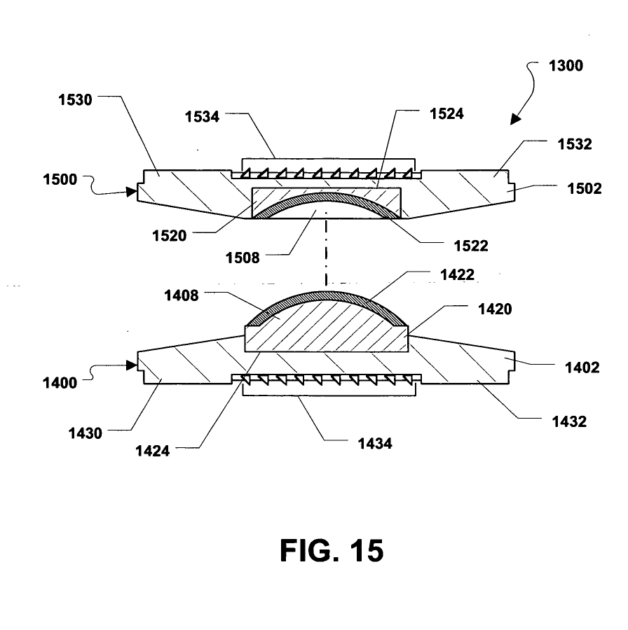

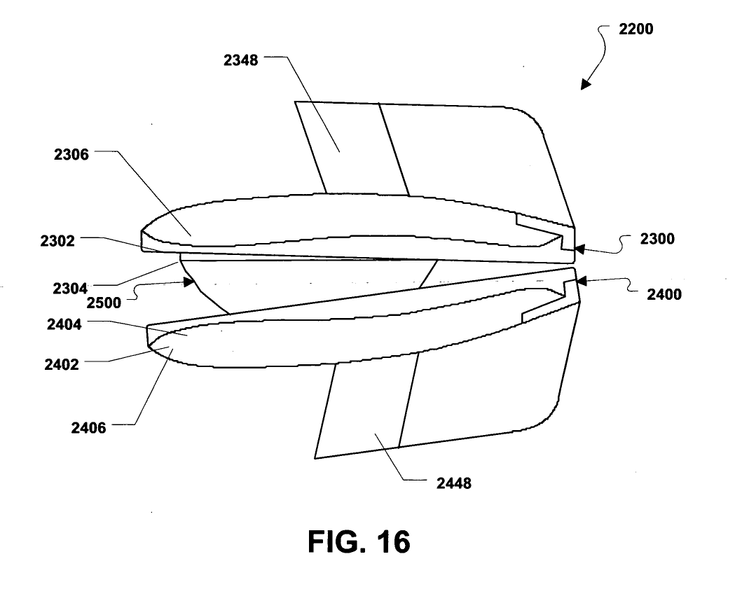

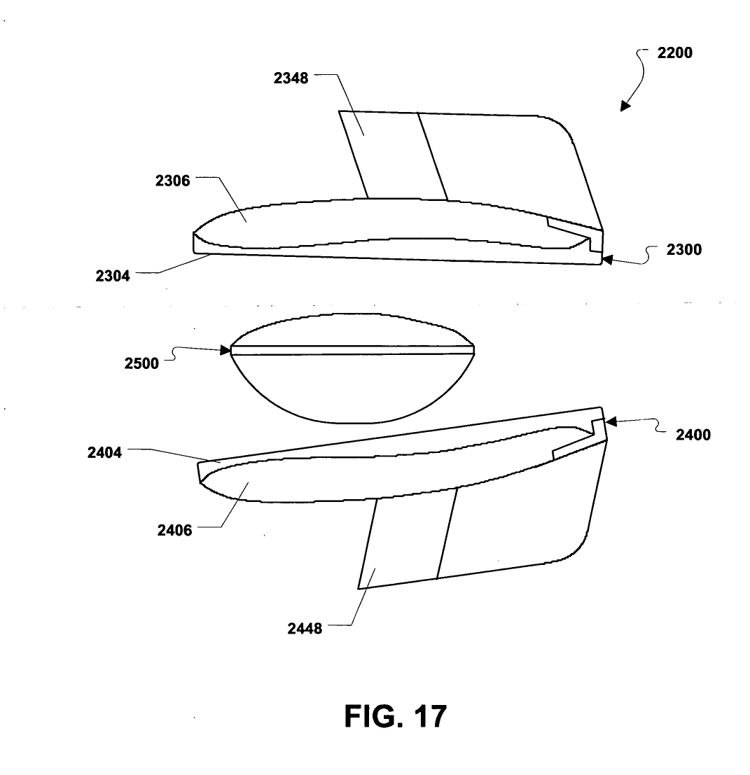

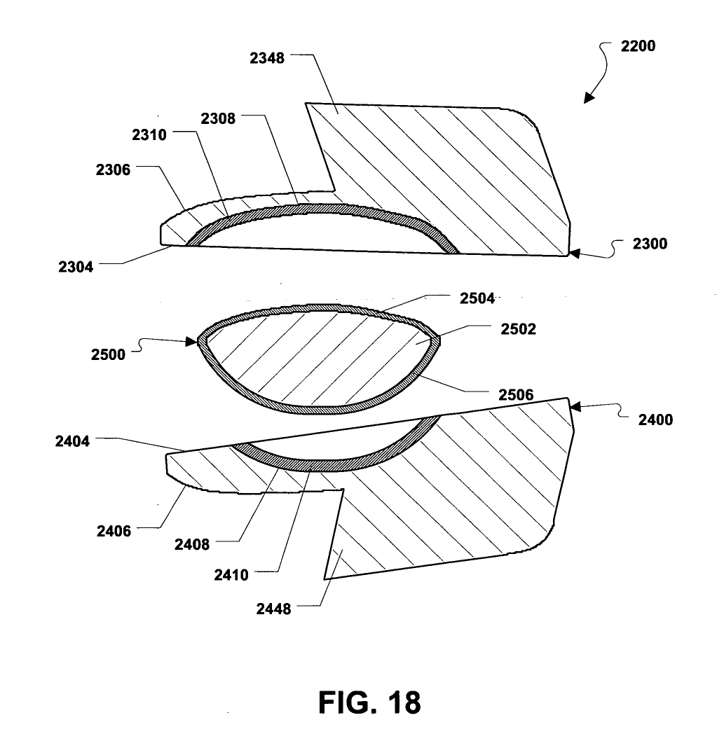

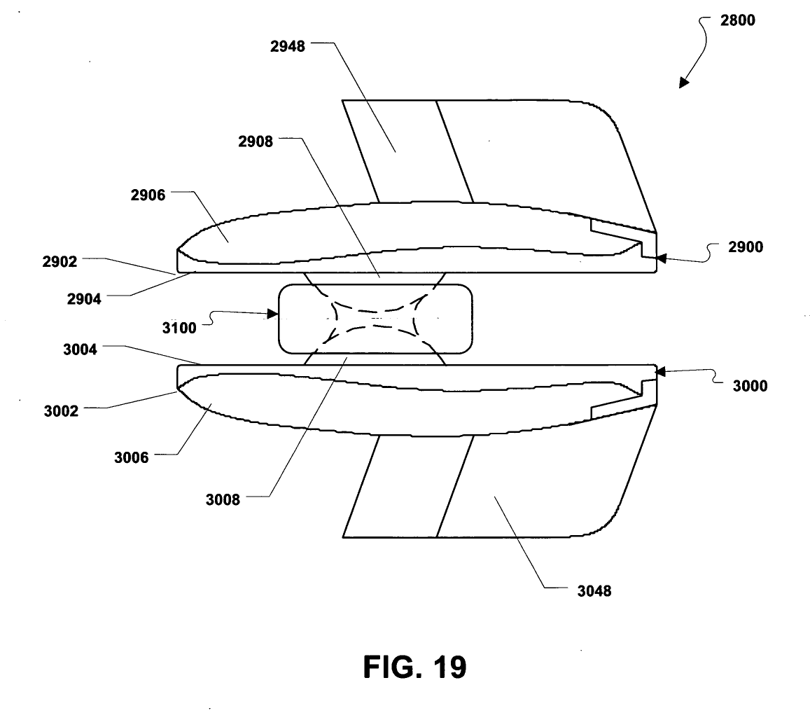

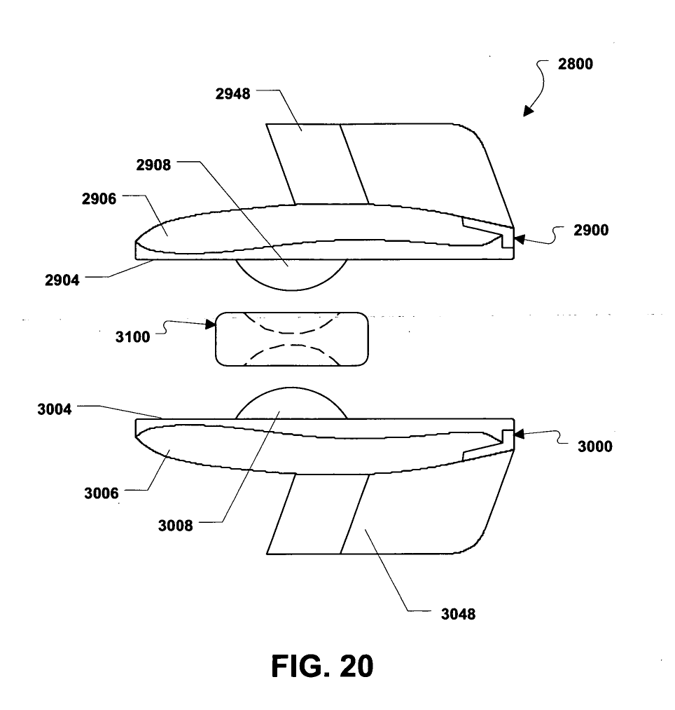

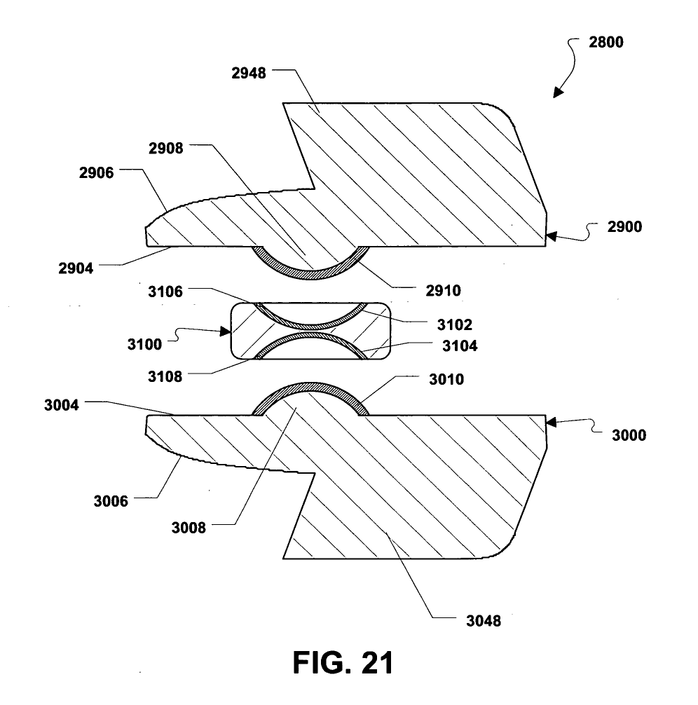

Referring to FIGS. 13 through 15, a second embodiment of an intervertebral prosthetic disc is shown and is generally designated 1300. As illustrated, the intervertebral prosthetic disc 1300 can include an inferior component 1400 and a superior component 1500. In a particular embodiment, the components 1400, 1500 can be made from one or more biocompatible materials. For example, the materials can be metal containing materials, polymer containing materials, or any combination thereof. In a particular embodiment, the one or both of the components 1400 and 1500 can be formed of a polymeric material including a rigid-rod polymer.

In a particular embodiment, the inferior component 1400 can include an inferior support plate 1402 that has an inferior articular surface 1404 and an inferior bearing surface 1406. In a particular embodiment, the inferior articular surface 1404 can be generally rounded and the inferior bearing surface 1406 can be generally flat.

As illustrated in FIG. 13 through FIG. 15, a projection 1408 extends from the inferior articular surface 1404 of the inferior support plate 1402. In a particular embodiment, the projection 1408 can have a hemi-spherical shape. Alternatively, the projection 1408 can have an elliptical shape, a cylindrical shape, or other arcuate shape.

The projection 1408 can be configure to movably engage a recession 1508 in the superior component 1500. For example, the recession 1508 can be configured to receive a hemi-spherical shaped projection, or alternatively, can be configured to receive an elliptical shaped projection, a cylindrical shaped projection, or another arcuate shaped projection.

Referring to an embodiment illustrated in FIG. 15, the projection 1408 can include a base 1420 and an inferior wear resistant layer 1422 affixed to, deposited on, or otherwise disposed on, the base 1420. In a particular embodiment, the base 1420 can act as a substrate and the inferior wear resistant layer 1422 can be deposited on the base 1420. Further, the base 1420 can engage a cavity 1424 that can be formed in the inferior support plate 1402. In a particular embodiment, the cavity 1424 can be sized and shaped to receive the base 1420 of the projection 1408. Further, the base 1420 of the projection 1408 can be press fit into the cavity 1424. Alternatively, the component 1400, the base 1420 and the superior wear resistant layer 1422 can be integrally formed of a single component or can be co-molded.

In addition, the recession 1508 can be formed by a superior base 1520. In an example, the superior base 1520 includes a superior wear resistant layer 1522. In an example, the superior base 1520 can be press fit into a cavity 1524 of the superior component 1500. Alternatively, the component 1500, the base 1520 and the superior wear resistant layer 1522 can be integrally formed of a single component or can be co-molded.

In a particular embodiment, the base 1420 of the projection can include a polymer material, such as an elastomeric material. In another example, the base 1420 can include a polymeric material including a rigid-rod polymer. Further, in a particular embodiment, the inferior wear resistant layer 1422 can be formed of a polymer material, such as a polymeric material including a rigid-rod polymer. For example, the inferior wear resistant layer 1422 can be formed essentially of a rigid-rod polymer material and placed on the base 1420. In an embodiment, the polymer material can be placed using conventional bonding, fastening, or deposition techniques. In a further example, the base 1420 and the inferior wear resistant layer 1422 can be co-molded.

As such, the base 1420 can be formed of a material that can allow inferior wear resistant layer 1422 to be placed or formed thereon. The base 1420 can be fitted into an inferior support plate 1402 made from one or more of the materials described herein. Alternatively, the inferior support plate. 1402, the base 1420, and the inferior wear resistant layer 1422 can be integrally formed of a single material or can be co-molded from different materials.

Also, in a particular embodiment, the base 1420 can be roughened prior to placement or formation of the inferior wear resistant layer 1422 thereon. For example, the base 1420 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 1420 on which the inferior wear resistant layer 1422 is placed can be serrated and can include one or more teeth, spikes, or other protrusions extending therefrom. The serrations of the base 1420 can facilitate anchoring of the inferior wear resistant layer 1422 on the base 1420 and can substantially reduce the likelihood of delamination of the inferior wear resistant layer 1422 from the base 1420.

In addition, the superior base 1520 can include a polymer material, such as an elastomeric material. In another example, the superior base 1520 can include a polymeric material including a rigid-rod polymer. Further, in a particular embodiment, the superior wear resistant layer 1522 can be formed of a polymer material, such as a polymeric material including a rigid-rod polymer. For example, the superior wear resistant layer 1522 can be formed essentially of a rigid-rod polymer material and placed on the superior base 1520. In an embodiment, the polymer material can be placed using conventional bonding, fastening, or deposition techniques. In a further example, the superior base 1520 and the superior wear resistant layer 1522 can be co-molded.

In a particular embodiment, the inferior wear resistant layer 1422 or the superior wear resistant layer 1522 can have a thickness in a range of fifty micrometers to five millimeters (50 μm-5 mm). Further, the inferior wear resistant layer 1422 or the superior wear resistant layer 1522 can have a thickness in a range of two hundred micrometers to two millimeters (200 μm-2 mm). In a particular embodiment, the serrations that can be formed on the surface of the base 1420 or of the superior base 1520 can have a height that is at most half of the thickness of the inferior wear resistant layer 1422 or of the superior wear resistant layer 1522. Accordingly, the likelihood that the serrations will protrude through the inferior wear resistant layer 1422 or through the superior wear resistant layer 1522 is substantially minimized.

Additionally, in a particular embodiment, a Young's modulus of the wear resistant layers 1422 or 1522 can be substantially greater than a Young's modulus of the base layers 1420 or 1520. Also, a hardness of the wear resistant layers 1422 or 1522 can be substantially greater than a hardness of the bases layers 1420 or 1520. Further, a toughness of the wear resistant layers 1422 or 1522 can be substantially greater than a toughness of the base layers 1420 or 1520. In a particular embodiment, the wear resistant layers 1422 or 1522 can be annealed immediately after deposition in order to minimize cracking of the inferior wear resistant layer. Also, the wear resistant layers 1422 or 1522 can be polished in order to minimize surface irregularities of the wear resistant layers 1422 or 1522 and increase a smoothness of the wear resistant layers 1422 or 1522.

According to a particular embodiment, the inferior wear resistant layer 1422 or the superior wear resistant layer 1522 can be formed of a polymeric material, such as a polymeric material including a rigid-rod polymer. In particular, the inferior wear resistant layer 1422 or the superior wear resistant layer 1522 can be formed essentially of a rigid-rod polymer matrix and can be essentially free of a filler material. It will be appreciated that in addition to the wear resistant layers provided herein, other components, such as, for example, the base components, can include a rigid-rod polymer material. In fact, according to an embodiment, the superior component and inferior component can be single component, molded pieces, consisting essentially of a rigid-rod polymer material.

FIG. 13 through FIG. 15 also show that the inferior component 1400 can include a first inferior keel 1430, a second inferior keel 1432, and a plurality of inferior teeth 1434 that extend from the inferior bearing surface 1406. Similarly, the superior component 1500 can include a first superior keel 1530, a second superior keel 1532, and a plurality of superior teeth 1534 that extend from the superior bearing surface 1506. As shown, in a particular embodiment, the keels 1430, 1432, 1530, or 1532 and the teeth 1434 or 1534 are generally saw-tooth, or triangle, shaped. Further, the keels 1430, 1432, 1530, or 1532 and the teeth 1434 or 1534 are designed to engage cancellous bone, cortical bone, or a combination thereof of an inferior vertebra. Additionally, the teeth 1434 or 1534 can prevent the component 1400 or 1500 from moving with respect to an associated vertebra after the intervertebral prosthetic disc 1300 is installed within the intervertebral space between the inferior vertebra and the superior vertebra.

In a particular embodiment, the teeth 1434 or 1534 can include other projections, such as spikes, pins, blades, or a combination thereof that have any cross-sectional geometry. In a particular example, the keels 1430, 1432, 1530, or 1532 and the teeth 1434 or 1534 can be formed of a polymeric material, such as a polymeric material including a rigid-rod polymer.

### Description of a Third Embodiment of an Intervertebral Prosthetic Disc

Referring to FIGS. 16 through 18, a third embodiment of an intervertebral prosthetic disc is shown and is generally designated 2200. As illustrated, the intervertebral prosthetic disc 2200 can include a superior component 2300, an inferior component 2400, and a nucleus 2500 disposed, or otherwise installed, there between. In a particular embodiment, the components 2300, 2400 and the nucleus 2500 can be made from one or more biocompatible materials. For example, the materials can be metal containing materials, polymer materials, or combinations thereof. Additionally, the biocompatible materials can include, or contain, an inorganic carbon-based material, such as graphite. In a particular embodiment, the metal containing materials can be metal. For example, the materials can be metal containing materials, polymer materials, or composite materials that include metals, polymers, or combinations of metals and polymers. The metal containing materials can be pure metals, metal alloys, or a metal containing a polymer or ceramic filler. The pure metals can include titanium. The metal alloys can include stainless steel, a cobalt-chrome-molybdenum alloy, e.g., ASTM F-999 or ASTM F-75, a titanium alloy, or a combination thereof.

In a particular embodiment, the components 2300, 2400 or 2500 can include a polymer material, such as a polymeric material including a rigid-rod polymer. In a particular embodiment, the components 2300, 2400, or 2500 can be formed essentially of a rigid-rod polymer material, such as a rigid-rod polymer material that is substantially free of fillers.

In a particular embodiment, the superior component 2300 can include a superior support plate 2302 that has a superior articular surface 2304 and a superior bearing surface 2306. In a particular embodiment, the superior articular surface 2304 can be substantially flat and the superior bearing surface 2306 can be generally curved. In an alternative embodiment, at least a portion of the superior articular surface 2304 can be generally curved and the superior bearing surface 2306 can be substantially flat.

In a particular embodiment, after installation, the superior bearing surface 2306 can be in direct contact with vertebral bone, e.g., cortical bone and cancellous bone. Further, the superior bearing surface 2306 can be coated with a bone-growth promoting substance, e.g., a hydroxyapatite coating formed of calcium phosphate. Additionally, the superior bearing surface 2306 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 18, a superior depression 2308 is established within the superior articular surface 2304 of the superior support plate 2302. In a particular embodiment, the superior depression 2308 can have an arcuate shape. For example, the superior depression 2308 can have a hemispherical shape, an elliptical shape, a cylindrical shape, or any combination thereof.

FIG. 16 through FIG. 18 indicate that the superior component 2300 can include a superior keel 2348 that extends from superior bearing surface 2306. During installation, described below, the superior keel 2348 can at least partially engage a keel groove that can be established within a cortical rim of a superior vertebra. Further, the superior keel 2348 can be coated with a bone-growth promoting substance, e.g., a hydroxyapatite coating formed of calcium phosphate. Additionally, the superior keel 2348 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 2400 can include an inferior support plate 2402 that has an inferior articular surface 2404 and an inferior bearing surface 2406. In a particular embodiment, the inferior articular surface 2404 can be substantially flat and the inferior bearing surface 2406 can be generally curved. In an alternative embodiment, at least a portion of the inferior articular surface 2404 can be generally curved and the inferior bearing surface 2406 can be substantially flat.

In a particular embodiment, after installation, the inferior bearing surface 2406 can be in direct contact with vertebral bone, e.g., cortical bone and cancellous bone. Further, the inferior bearing surface 2406 can be coated with a bone-growth promoting substance, e.g., a hydroxyapatite coating formed of calcium phosphate. Additionally, the inferior bearing surface 2406 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 18, an inferior depression 2408 is established within the inferior articular surface 2404 of the inferior support plate 2402. In a particular embodiment, the inferior depression 2408 can have an arcuate shape. For example, the inferior depression 2408 can have a hemispherical shape, an elliptical shape, a cylindrical shape, or any combination thereof.

FIGS. 16-18 indicate that the inferior component 2400 can include an inferior keel 2448 that extends from inferior bearing surface 2406. During installation, described below, the inferior keel 2448 can at least partially engage a keel groove that can be established within a cortical rim of a vertebra. Further, the inferior keel 2448 can be coated with a bone-growth promoting substance, e.g., a hydroxyapatite coating formed of calcium phosphate. Additionally, the inferior keel 2448 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular example, the superior component 2300 or the inferior component 2400 can be formed as an integral component of a polymeric material, such as a polymeric material including a rigid-rod polymer. In the example illustrated in FIG. 18, the superior depression 2308 or the inferior depression 2408 can include a wear resistant layer 2310 or 2410. The wear resistant layer 2310 or 2410 can be coated or adhered to the component 2300 or 2400. Alternatively, the component 2300 or 2400 can be co-molded with the wear resistant layer 2310 or 2410.

As illustrated in FIG. 16, FIG. 17, and FIG. 18, the nucleus 2500 is configured to engage the depressions 2308 or 2408 of the components 2300 or 2400. As illustrated in FIG. 18, the nucleus 2500 can include a core 2502. In an example, a superior wear resistant layer 2504 can be deposited on, or affixed to, the core 2502. In another example, an inferior wear resistant layer 2506 can be deposited on, or affixed to, the core 2502. In a particular embodiment, the core 2502 can include a polymer material, such as an elastomeric material or a polymeric material including a rigid-rod polymer. In another example, the wear resistant layer 2504 or 2506 can be formed of a polymeric material, such as an elastomeric material or a polymeric material including a rigid-rod polymer. In a particular example, the polymeric material can consist essentially of a rigid-rod polymer and can be substantially free of filler. In a further exemplary embodiment, a core 2502 of the nucleus 2500 can be formed of an elastomeric polymer material and the wear resistant layers 2504 or 2506 can be formed of an polymeric material including a rigid-rod polymer, such as a rigid-rod polymer substantially free of filler.

Additionally, the superior wear resistant layer 2504 and the inferior wear resistant layer 2506 can each have an arcuate shape. For example, the superior wear resistant layer 2504 of the nucleus 2500 and the inferior wear resistant layer 2506 of the nucleus 2500 can have a hemispherical shape, an elliptical shape, a cylindrical shape, or any combination thereof. Further, in a particular embodiment, the superior wear resistant layer 2504 can be curved to match the superior depression 2308 of the superior component 2300. Also, in a particular embodiment, the inferior wear resistant layer 2506 of the nucleus 2500 can be curved to match the inferior depression 2408 of the inferior component 2400.

As illustrated in FIG. 16, the superior wear resistant layer 2504 of the nucleus 2500 can engage the superior wear resistant layer 2310 within the superior depression 2308 and can allow relative motion between the superior component 2300 and the nucleus 2500. Also, the inferior wear resistant layer 2506 of the nucleus 2500 can engage the inferior wear resistant layer 2410 within the inferior depression 2408 and can allow relative motion between the inferior component 2400 and the nucleus 2500. Accordingly, the nucleus 2500 can engage the superior component 2300 and the inferior component 2400 and the nucleus 2500 can allow the superior component 2300 to rotate with respect to the inferior component 2400.

In a particular embodiment, the overall height of the intervertebral prosthetic device 2200 can be in a range from fourteen millimeters to forty-six millimeters (14-46 mm). Further, the installed height of the intervertebral prosthetic device 2200 can be in a range from eight millimeters to sixteen millimeters (8-16 mm). In a particular embodiment, the installed height can be substantially equivalent to the distance between an inferior vertebra and a superior vertebra when the intervertebral prosthetic device 2200 is installed there between.

In a particular embodiment, the length of the intervertebral prosthetic device 2200, e.g., along a longitudinal axis, can be in a range from thirty millimeters to forty millimeters (30-40 mm). Additionally, the width of the intervertebral prosthetic device 2200, e.g., along a lateral axis, can be in a range from twenty-five millimeters to forty millimeters (25-40 mm).

### Description of a Fourth Embodiment of an Intervertebral Prosthetic Disc

Referring to FIGS. 19 through 21, a fourth embodiment of an intervertebral prosthetic disc is shown and is generally designated 2800. As illustrated, the intervertebral prosthetic disc 2800 can include a superior component 2900, an inferior component 3000, and a nucleus 3100 disposed, or otherwise installed, therebetween. In a particular embodiment, the components 2900, 3000 and the nucleus 3100 can be made from one or more biocompatible materials. For example, the materials can be metal containing materials, polymer materials, or combinations thereof. Additionally, the biocompatible materials can include, or contain, an inorganic carbon-based material, such as graphite. In a particular embodiment, the materials can be metal containing materials, polymer materials, or combinations thereof. Further, for example, the metal containing materials can be pure metals, metal alloys, or a metal containing a polymer or ceramic filler. The pure metals can include titanium. The metal alloys can include stainless steel, a cobalt-chrome-molybdenum alloy, e.g., ASTM F-999 or ASTM F-75, a titanium alloy, or a combination thereof.

In a particular embodiment, the components 2900, 3000 or 3100 can include a polymer material, such as a polymeric material including a rigid-rod polymer. In a particular embodiment, the components 2900, 3000, or 3100 can be formed essentially of a rigid-rod polymer material, such as a rigid-rod polymer material that is substantially free of fillers.

In a particular embodiment, the superior component 2900 can include a superior support plate 2902 that has a superior articular surface 2904 and a superior bearing surface 2906. In a particular embodiment, the superior articular surface 2904 can be substantially flat and the superior bearing surface 2906 can be generally curved. In an alternative embodiment, at least a portion of the superior articular surface 2904 can be generally curved and the superior bearing surface 2906 can be substantially flat.

In a particular embodiment, after installation, the superior bearing surface 2906 can be in direct contact with vertebral bone, e.g., cortical bone and cancellous bone. In addition, the superior component 2900 can include a superior keel 2948 that extends from superior bearing surface 2906. Further, the superior bearing surface 2906 or the superior keel 2948 can be coated with a bone-growth promoting substance, e.g., a hydroxyapatite coating formed of calcium phosphate. Additionally, the superior bearing surface 2906 or the superior keel 2948 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 19 through FIG. 21, a superior projection 2908 extends from the superior articular surface 2904 of the superior support plate 2902. In a particular embodiment, the superior projection 2908 can have an arcuate shape. For example, the superior depression 2908 can have a hemispherical shape, an elliptical shape, a cylindrical shape, or any combination thereof.

In a particular embodiment, the inferior component 3000 can include an inferior support plate 3002 that has an inferior articular surface 3004 and an inferior bearing surface 3006. In a particular embodiment, the inferior articular surface 3004 can be substantially flat and the inferior bearing surface 3006 can be generally curved. In an alternative embodiment, at least a portion of the inferior articular surface 3004 can be generally curved and the inferior bearing surface 3006 can be substantially flat.

After installation, the inferior bearing surface 3006 can be in direct contact with vertebral bone, e.g., cortical bone and cancellous bone. In addition, the inferior component 3000 can include an inferior keel 3048 that extends from inferior bearing surface 3006. Further, the inferior bearing surface 3006 or the inferior keel 3048 can be coated with a bone-growth promoting substance, e.g., a hydroxyapatite coating formed of calcium phosphate. Additionally, the inferior bearing surface 3006 or the inferior keel 3048 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 19 through FIG. 21, an inferior projection 3008 can extend from the inferior articular surface 3004 of the inferior support plate 3002. In a particular embodiment, the inferior projection 3008 can have an arcuate shape. For example, the inferior projection 3008 can have a hemispherical shape, an elliptical shape, a cylindrical shape, or any combination thereof.

FIG. 21 shows that the superior projection 2908 or that the inferior projection 3008 can include a superior wear resistant layer 2910 or an inferior wear resistant layer 3010, respectively. In a particular embodiment, the superior wear resistant layer 2910 or the inferior wear resistant layer 3010 can be attached to, affixed to, or otherwise deposited on, the superior projection 2908 or the inferior projection 3008. In a particular embodiment, the superior wear resistant layer 2910 or the inferior wear resistant layer 3010 can be formed of a polymeric material including a rigid-rod polymer. For example, the polymeric material can be essentially rigid-rod polymer and can be substantially free of filler.

Further, FIG. 21 shows that the nucleus 3100 can include a superior depression 3102 and an inferior depression 3104. In a particular embodiment, the superior depression 3102 and the inferior depression 3104 can each have an arcuate shape. For example, the superior depression 3102 of the nucleus 3100 and the inferior depression 3104 of the nucleus 3100 can have a hemispherical shape, an elliptical shape, a cylindrical shape, or any combination thereof. In a particular embodiment, the superior depression 3102 can be curved to match the superior projection 2908 of the superior component 2900. Also, in a particular embodiment, the inferior depression 3104 of the nucleus 3100 can be curved to match the inferior projection 3008 of the inferior component 3000.

As illustrated in FIG. 21, a superior wear resistant layer 3106 can be disposed within, or deposited within, the superior depression 3102 of the nucleus 3100. Also, an inferior wear resistant layer 3108 can be disposed within, or deposited within, the inferior depression 3103 of the nucleus 3100. In a particular embodiment, the superior wear resistant layer 3106 and the inferior wear resistant layer 3108 can be formed of a polymeric material, such as a polymeric material including a rigid-rod polymer. In particular, the superior wear resistant layer 3106 or the inferior wear resistant layer 3108 can be formed essentially of a rigid-rod polymer and can be substantially free of filler. In a further exemplary embodiment, a core of the nucleus 3100 can be formed of an elastomeric polymer material and the wear resistant layers 3106 or 3108 can be formed of an polymeric material including a rigid-rod polymer, such as a rigid-rod polymer substantially free of filler.

As illustrated in FIG. 19, the superior wear resistant layer 3106 of the nucleus 3100 can engage the superior wear resistant layer 2910 of the superior component 2900 and can allow relative motion between the superior component 2900 and the nucleus 3100. Also, the inferior wear resistant layer 3108 of the nucleus 3100 can engage the inferior wear resistant layer 3010 of the inferior component 3000 and can allow relative motion between the inferior component 3000 and the nucleus 3100. Accordingly, the nucleus 3100 can engage the superior component 2900 and the inferior component 3000, and the nucleus 3100 can allow the superior component 2900 to rotate with respect to the inferior component 3000.

In a particular embodiment, the overall height of the intervertebral prosthetic device 2800 can be in a range from fourteen millimeters to forty-six millimeters (14-46 mm). Further, the installed height of the intervertebral prosthetic device 2800 can be in a range from eight millimeters to sixteen millimeters (8-16 mm). In a particular embodiment, the installed height can be substantially equivalent to the distance between an inferior vertehra and a superior vertebra when the intervertebral prosthetic device 2800 is installed there between.

In a particular embodiment, the length of the intervertebral prosthetic device 2800, e.g., along a longitudinal axis, can be in a range from thirty millimeters to forty millimeters (30-40 mm). Additionally, the width of the intervertebral prosthetic device 2800, e.g., along a lateral axis, can be in a range from twenty-five millimeters to forty millimeters (25-40 mm).

### Description of a Nucleus Implant

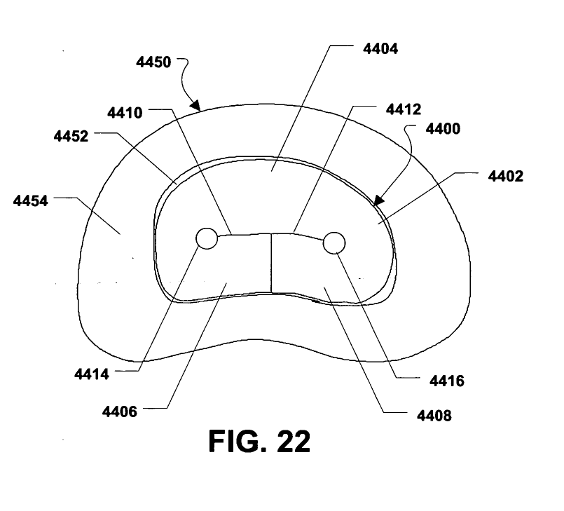

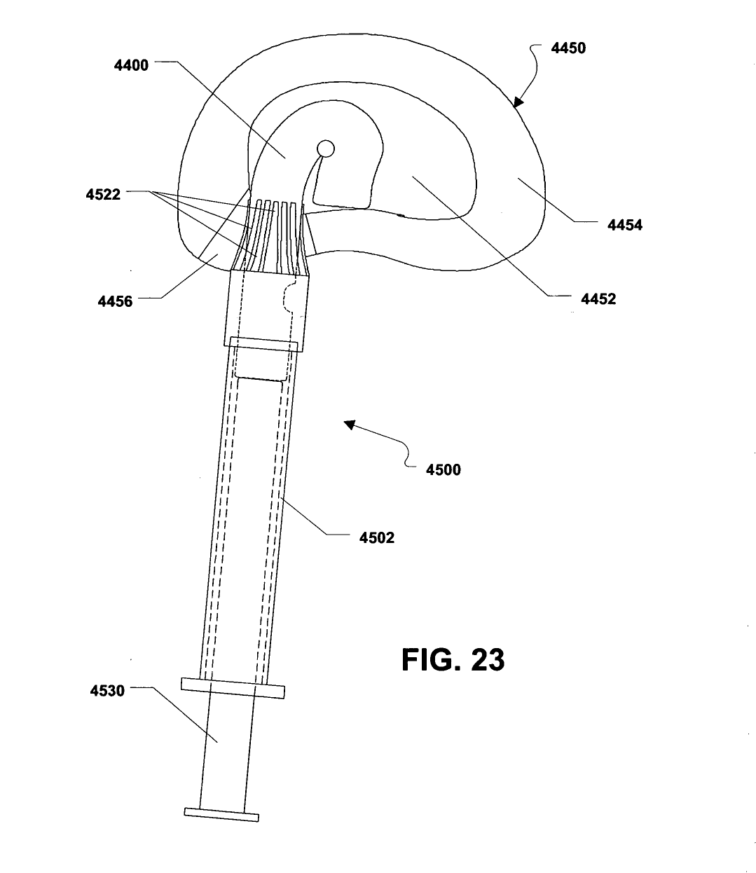

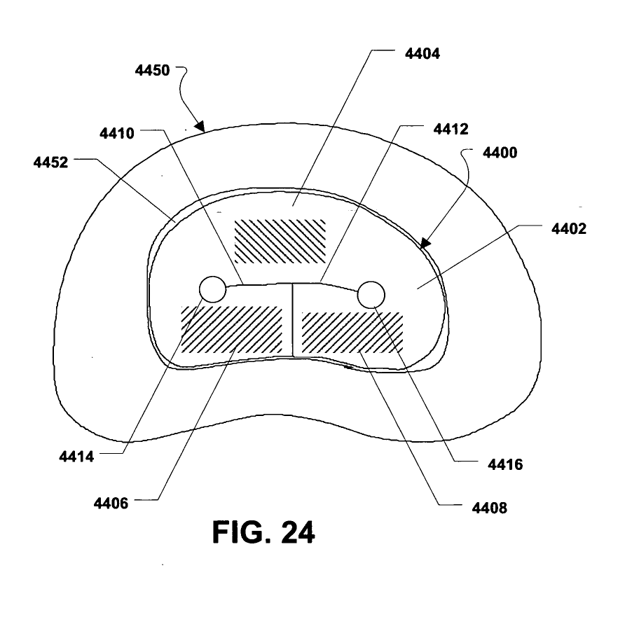

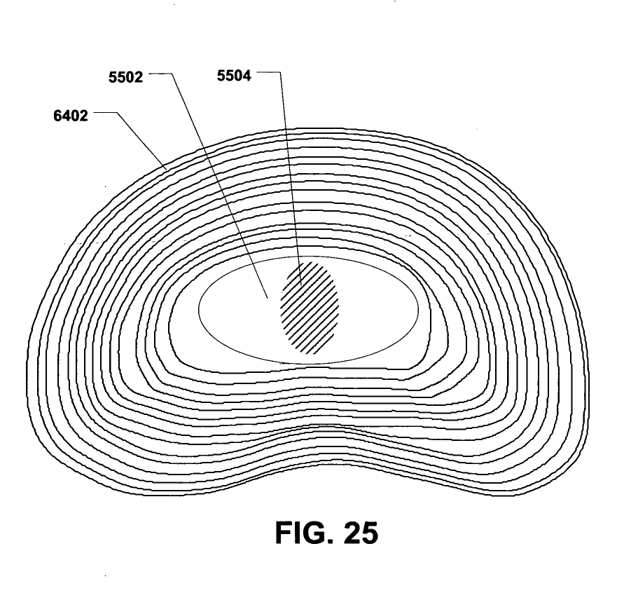

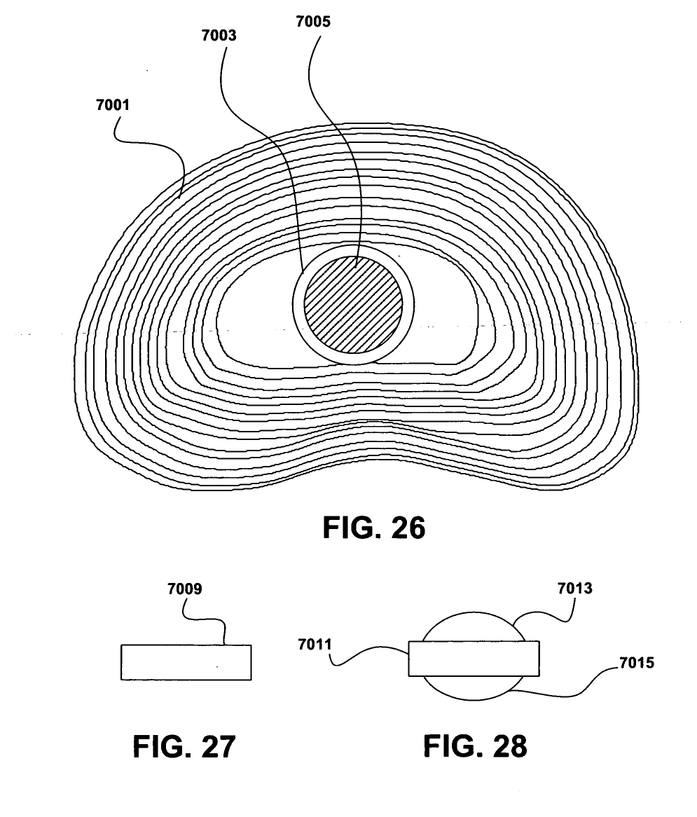

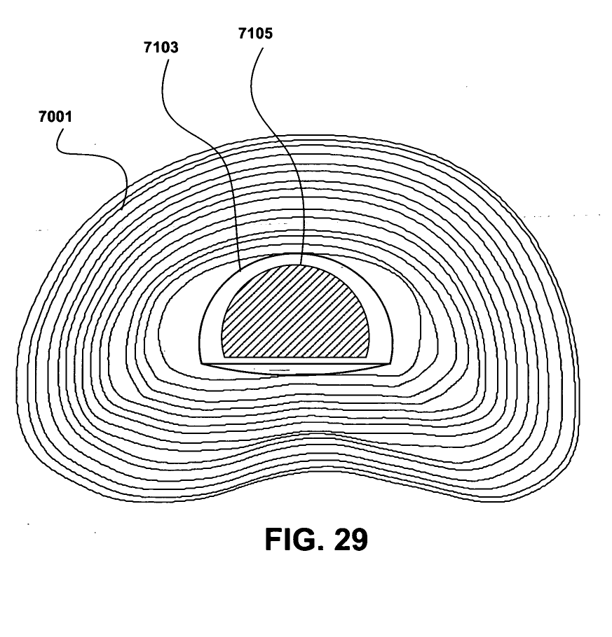

Referring to FIG. 22 through FIG. 24, an embodiment of a nucleus implant is shown and is designated 4400. As shown, the nucleus implant 4400 can include a load bearing elastic body 4402. The load bearing elastic body 4402 can include a central portion 4404. A first end 4406 and a second end 4408 can extend from the central portion 4404 of the load bearing elastic body 4402.

As depicted in FIG. 22, the first end 4406 of the load bearing elastic body 4402 can establish a first fold 4410 with respect to the central portion 4404 of the load bearing elastic body 4402. Further, the second end 4408 of the load bearing elastic body 4402 can establish a second fold 4412 with respect to the central portion 4404 of the load bearing elastic body 4402. In a particular embodiment, the ends 4406, 4408 of the load bearing elastic body 4402 can be folded toward each other relative to the central portion 4404 of the load bearing elastic body 4402. Also, when folded, the ends 4406, 4408 of the load bearing elastic body 4402 are parallel to the central portion 4404 of the load bearing elastic body 4402. Further, in a particular embodiment, the first fold 4410 can define a first aperture 4414 and the second fold 4412 can define a second aperture 4416. In a particular embodiment, the apertures 4414, 4416 are generally circular. However, the apertures 4414, 4416 can have any arcuate shape.

In an exemplary embodiment, the nucleus implant 4400 can have a rectangular cross-section with sharp or rounded corners. Alternatively, the nucleus implant 4400 can have a circular cross-section. As such, the nucleus implant 4400 may form a rectangular prism or a cylinder.

FIG. 22 indicates that the nucleus implant 4400 can be implanted within an intervertebral disc 4450 between a superior vertebra and an inferior vertebra. More specifically, the nucleus implant 4400 can be implanted within an intervertebral disc space 4452 established within the annulus fibrosis 4454 of the intervertebral disc 4450. The intervertebral disc space 4452 can be established by removing the nucleus pulposus (not shown) from within the annulus fibrosis 4454.

In a particular embodiment, the nucleus implant 4400 can provide shock-absorbing characteristics substantially similar to the shock absorbing characteristics provided by a natural nucleus pulposus. Additionally, in a particular embodiment, the nucleus implant 4400 can have a height that is sufficient to provide proper support and spacing between a superior vertebra and an inferior vertebra.

In particular, the nucleus implant 4400 illustrated in FIG. 22 can have a shape memory and the nucleus implant 4400 can be configured to allow extensive short-term manual, or other, deformation without permanent deformation, cracks, tears, breakage or other damage, that can occur, for example, during placement of the implant into the intervertebral disc space 4452.

For example, the nucleus implant 4400 can be deformable, or otherwise configurable, e.g., manually, from a folded configuration, shown in FIG. 22, to a substantially straight configuration, in which the ends 4406, 4408 of the load bearing elastic body 4402 are substantially aligned with the central portion 4404 of the load bearing elastic body 4402. In a particular embodiment, when the nucleus implant 4400 the folded configuration, shown in FIG. 22, can be considered a relaxed state for the nucleus implant 4400. Also, the nucleus implant 4400 can be placed in the straight configuration for placement, or delivery into an intervertebral disc space within an annulus fibrosis.

In a particular embodiment, the nucleus implant 4400 can include a shape memory, and as such, the nucleus implant 4400 can automatically return to the folded, or relaxed, configuration from the straight configuration after force is no longer exerted on the nucleus implant 4400. Accordingly, the nucleus implant 4400 can provide improved handling and manipulation characteristics since the nucleus implant 4400 can be deformed, configured, or otherwise handled, by an individual without resulting in any breakage or other damage to the nucleus implant 4400.

Although the nucleus implant 4400 can have a wide variety of shapes, the nucleus implant 4400 when in the folded, or relaxed, configuration can conform to the shape of a natural nucleus pulposus. As such, the nucleus implant 4400 can be substantially elliptical when in the folded, or relaxed, configuration. In one or more alternative embodiments, the nucleus implant 4400, when folded, can be generally annular-shaped or otherwise shaped as required to conform to the intervertebral disc space within the annulus fibrosis. Moreover, when the nucleus implant 4400 is in an unfolded, or non-relaxed, configuration, such as the substantially straightened configuration, the nucleus implant 4400 can have a wide variety of shapes. For example, the nucleus implant 4400, when straightened, can have a generally elongated shape. Further, the nucleus implant 4400 can have a cross section that is: generally elliptical, generally circular, generally rectangular, generally square, generally triangular, generally trapezoidal, generally rhombic, generally quadrilateral, any generally polygonal shape, or any combination thereof.

Referring to FIG. 23, a nucleus delivery device is shown and is generally designated 4500. The elongated housing 4502 can be hollow and can form an internal cavity. FIG. 23 further shows that the nucleus delivery device 4500 can include a generally elongated plunger. In a particular embodiment, the plunger 4530 can be sized and shaped to slidably fit within the housing 4502, e.g., within the cavity of the housing 4502.

As shown in FIG. 23, a nucleus implant, e.g., the nucleus implant 4400 shown in FIG. 22, can be disposed within the housing 4502, e.g., within the cavity of the housing 4502. Further, the plunger 4530 can slide within the cavity, relative to the housing 4502, in order to force the nucleus implant 4400 from within the housing 4502 and into the intervertebral disc space 4452. As shown in FIG. 23, as the nucleus implant 4400 exits the nucleus delivery device 4500, the nucleus implant 4400 can move from the non-relaxed, straight configuration to the relaxed, folded configuration within the annulus fibrosis. Further, as the nucleus implant 4400 exits the nucleus delivery device 4500, the nucleus implant 4400 can cause movable members 4522 to move to the open position, as shown in FIG. 23.

In a particular embodiment, the nucleus implant 4400 can be installed using a posterior surgical approach, as shown. Further, the nucleus implant 4400 can be installed through a posterior incision 4456 made within the annulus fibrosis 4454 of the intervertebral disc 4450. Alternatively, the nucleus implant 4400 can be installed using an anterior surgical approach, a lateral surgical approach, or any other surgical approach.

Referring to FIG. 24, the load bearing elastic body 4402 is illustrated as including a first end 4406, a second end 4408, and a central region 4404. In a particular embodiment, the polymeric material at the first end 4406 and at the second end 4408 can include a rigid-rod polymer, such as at the surface of the first end 4406 or the second end 4408. In another example, the polymeric material at the central portion 4404 can include a rigid-rod polymer, such as at the surface of the central portion 4404. Alternatively, the load bearing elastic body 4402 can include a polymeric material including a rigid-rod polymer. In a particular example, the load bearing elastic body 4402 can be formed of an elastomeric polymer and can be coated on a top surface and a bottom surface with a rigid-rod polymer material.

In another example illustrated in FIG. 25, a load bearing elastic body, such as a load bearing body 5502 can be inserted between two vertebrae into a region formerly occupied by the nucleus pulposus 6404 and surrounded by the annulus fibrosis. In the embodiment illustrated in FIG. 25, the load bearing body 5502 can have an elliptical shape. Alternatively, the load bearing body 5502 can have a spheroidal shape, an ellipsoidal shape, a cylindrical shape, a polygonal prism shape, a tetrahedral shape, a frustoconical shape, or any combination thereof. In a particular embodiment, the load bearing body 5502 can include a stabilizer, such as a stabilizer in the shape of a disc extending radially from an axially central location of the load bearing body.

In an exemplary embodiment, the load bearing body 5502 illustrated in FIG. 25 can have a maximum radius that is greater than the distance between the two vertebrae between which the load bearing body is to be implanted. Alternatively, the maximum radius can be equal to or less than the distance between the two vertebrae between which the load bearing body 5502 is to be implanted. In a particular embodiment, the maximum diameter of the load bearing body can be between about 5 mm to about 35 mm, such as about 10 mm to about 30 mm.

In a particular embodiment, the load bearing body 5502 is formed of a polymeric material. In an example, the polymeric material can include a rigid-rod polymer. In another example, the polymeric material can include an elastomeric material that is at least partially coated with a rigid-rod polymer. For example, the load bearing body 5502 can be coated in a center portion 5504, as illustrated in FIG. 25. Alternatively, the load bearing body 5502 can be coated at a left portion, a right portion, an anterior portion, a posterior portion, a top portion, a bottom portion, or any combination thereof. In a particular example, the load bearing body 5502 can be formed of an elastomeric material and can be coated on a top surface and on a bottom surface with a rigid-rod polymer material. In another example, the load bearing body 5502 can be formed of a material having a modulus less than the modulus of a rigid-rod polymer coating material.

While the above embodiments of prosthetic disc replacement devices and nucleus devices have been discussed in relation to implants for the location in the intervertebral space, additional embodiments can be envisioned for location in proximity to the zygapophysial joint, such as between articular processes.

In another example illustrated in FIG. 26, a load bearing body having an outer portion 7003 is illustrated. As previously described the load bearing body can be configured to be installed between two vertebrae into a region formerly occupied by the nucleus pulposus and surrounded by the annulus fibrosis 7001. According to an embodiment illustrated in FIG. 26, the load bearing body can have an spherical contour, particularly the outer portion 7003 can have a spherical contour. As such, the load bearing body can also include a central portion 7005 that can have the same or similar shape to the outer portion 7003 of the load bearing body. Alternatively, the load bearing body 7003 can have a less spherical contour, such as a circular contour with a low profile. Referring to FIG. 27, a cross section of a circular load bearing body 7009, similar to the one illustrated in FIG. 26, is provided. According to one embodiment, the load bearing body 7009 can include a low profile cross sectional contour, such as a disk-like contour, or the like. Alternatively, FIG. 28 provides another cross sectional illustration of a load bearing body 7011, which can include a disk-like portion and an upper hemispherical portion 7013 and a lower hemispherical portion 7015.

According to another exemplary embodiment, FIG. 29 illustrates a load bearing body having an outer portion 7103 and a central portion 7105 having a semi-asymmetric shape, such as a clam-shell contour or the like. Referring to FIG. 30, a load bearing body having an outer portion 7203 and a central portion 7205 having an elongated contour, resembling a pill or a generally rectangular portion with curved end sections.

In a particular embodiment, a nucleus implant can be formed essentially of a rigid-rod polymer. As described above, each of the components including intervertebral spacers and nucleus implants can include a rigid-rod polymer material and can be essentially free of filler material. Alternatively, the component can be formed of multiple material layers, such as a core material and a surface material. For example, the core material can be a polymeric material including a rigid-rod polymer. Alternatively, the core material can be formed of a material, such as a metallic, ceramic, or polymeric material, and the surface material can be formed of a rigid-rod polymer. In a further example, the core material can be formed of a polymeric material including a rigid-rod polymer and the surface material can be formed of a metallic, ceramic, or polymeric material, such as a diamond-like coating, ion-implanted coating, metal coating, ceramic coating, or any combination thereof. In a further exemplary embodiment, the component can include a layer formed of a first polymeric material including a rigid-rod polymer and a layer formed of a second polymeric material including a rigid-rod polymer.

It will also be appreciated that any of the wear resistant layers provided herein can include a rigid-rod polymer material that is suitable for articulating against another wear resistant layer of material including a metal, other polymer or ceramic. According to an embodiment, a wear resistant layer including a rigid-rod polymer material is configured to articulate against an adjacent wear resistant layer including a metal, such as titanium, titanium carbide, cobalt, chromium, metal alloys thereof, or other metal alloys. In another embodiment, a wear resistant layer including a rigid-rod polymer material is configured to articulate against an adjacent wear resistant layer including another polymer material, such as PAEK, PEEK, PEK, PEKK, UHMWPE, or the like. Still, according to another embodiment, a wear resistant layer including a rigid-rod polymer material is configured to articulate against an adjacent wear resistant layer including a ceramic, such as oxides, nitrides, carbides, other carbon-containing compounds, or the like.

Further, portions of components configured to fixably engage an osteal structure can be formed of a porous material, such as a porous rigid-rod polymer matrix. Such porous materials can include pores having pore size of about 10 microns to about 1000 microns, such as about 250 microns to about 750 microns. Further, the porous material can have a porosity of about 10% to about 50%. In addition, the porous material can be impregnated with an osteogenerative agent. For example, the osteogenerative agent can include hydroxyapatite and BMP. Treatment Kit

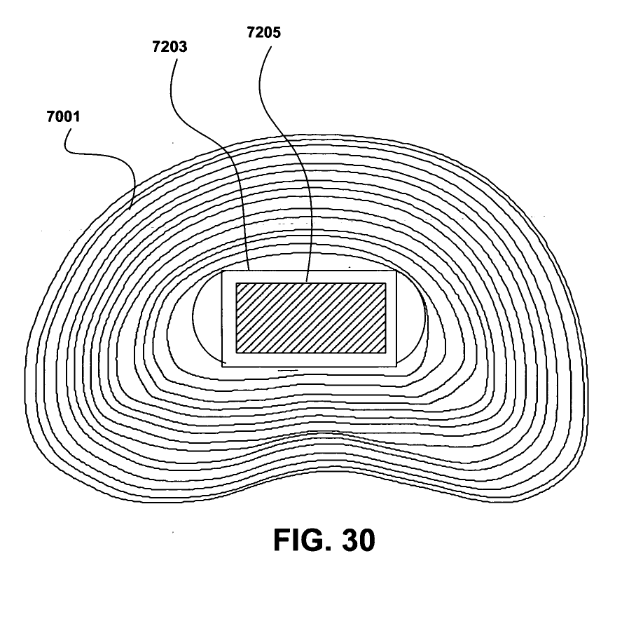

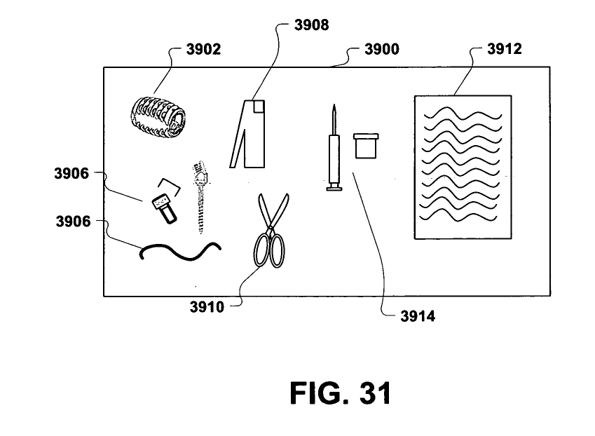

An implantable device described herein or components thereof can be included in a kit. In an exemplary embodiment, FIG. 31 includes an illustration of an exemplary kit 3900. For example, the kit 3900 can include a device component 3902. The device component 3902 can be adapted to engage a portion of the spine, such as a vertebra. In a particular example, the component 3902 can include a prosthetic disc, a nucleus implant, or any of the above described embodiments. In addition or alternatively, the kit 3900 can include a strand material 3904 or a fastener 3906 adapted to engage a joint, such as a zygapophysial joint, or a process, such as a spinous process or an articular process.

In addition, the kit 3900 can include a tool to further adapt the component 3902 or the strand material 3904, such as scissors 3910 or a cutting tool. For example the component 3902 or the strand material 3904 can be adapted based on the location or the size of the processes it is to engage.

In another example, the kit 3900 can include one or more fasteners 3906. For example, the kit 3900 can include staples, screws, or crimp fasteners to secure the component 3902 or the strand material 3904. In a further example, the kit 3900 can include a tool 3908 to secure the component 3902 or the strand material 3904. For example, the tool 3908 can be a stapler or a screwdriver to secure the component 3902 to a process or a vertebral body. In another example, the tool 3908 can include a crimp tool to secure the strand material 3904 or the component 3902 to itself.

In an additional example, the kit 3900 can include an agent 3914. For example, the kit 3900 can include an agent 3914 and a syringe for injecting the agent 3914 into the component 3902, or a portion of the spine. In another example, the syringe can include a gel that includes the agent 3914 for injection into a space proximate to the component 3902 and a portion of the spine. In an alternative embodiment, the syringe can include an adhesive, gel material, or bone cement to facilitate fusion of the component 3902 and a vertebra.

In a particular embodiment, the kit 3900 includes an indication of the use of the component 3902 or the strand material 3904. For example, an indicator 3912 can identify the kit 3900 as a repair or support system for a portion of the spine. In another example, the indicator 3912 can include contraindications for use of the kit 3900 and materials 3902 and 3904. In a further example, the indicator 3912 can include instructions, such as instructions regarding the installation of the device and materials 3902 and 3904.

In an exemplary embodiment, the kit components can be disposed in a closed container, which can be adequate to maintain the contents of the container therein during routine handling or transport, such as to a healthcare facility or the like.

### Method of Implanting

The implantable devices described herein can be generally implanted subcutaneously in proximity to or within the spine. For example, the implantable device can be implanted within an intervertebral space, within or across a zygapophysial joint, between spinous processes, or across the outer surface of two vertebra. To implant the device, a surgeon can approach the spine from one of several directions including posteriorally, through the abdomen, or laterally.

Generally, the implantable device includes at least one component. When the implantable device includes more than one component, the implantable device can be prepared by assembling the device. Alternatively, the device can be assembled as parts are engaged with the spine. In another example, the implantable device can be prepared by applying an agent to the device or impregnating the device with an agent. In a further example, the implantable device can be prepared by configuring the device, such as adjusting the size of the device.

For particular devices, the space between two vertebrae can be extended to permit insertion of the device. Alternatively, the device can be implanted and the implanted device can be extended to provide the desired spacing between vertebrae.

Once the device is implanted, a surgeon can remove tools used in the insertion process and close the surgical wound.

### CONCLUSION

With embodiments of the devices described above, the condition of a spine, and in particular, a set of discs and zygapophysial joints, can be maintained, repaired, or secured. Such a device can be used to limit further deterioration of a degrading of the spine.

In a particular embodiment, the device can act to restore movement of the processes and the associated vertebra relative to each other. As such, the device can reduce the likelihood of further injury to soft tissue associated with the spine, reduce pain associated with spine damage, and complement other devices.

Particular embodiments of the implantable device including a component formed of a polymeric material including a rigid-rod polymer can advantageously provide improved device performance. For example, a prosthetic disc device including a polymeric material including a rigid-rod polymer matrix can provide osteoconductive surfaces while also providing a strong structural support. Particular surfaces, such as wear resistant surfaces can be formed of a rigid-rod polymer material and can be polished to provide a low surface roughness. In addition, surfaces formed of particular rigid-rod polymer materials, such as homogeneous polymer blends and rigid-rod polymer materials that are free of filler, can provide surfaces that limit wear debris when subjected to friction.

Moreover, particular species of rigid-rod polymer provide a combination of advantageous-properties to polymeric-materials forming spinal implant-devices. In an exemplary embodiment, the rigid-rod polymer can be a thermoplastic rigid-rod polymer. In addition, particular rigid-rod polymers provide substantially isotropic mechanical properties. In particular, a polymeric material including a thermoplastic isotropic rigid-rod polymer, and particularly an amorphous thermoplastic isotropic rigid-rod polymer, can advantageously be used in components of an implantable device, alone or as a polymer matrix.

The above-disclosed subject matter is to be considered illustrative, and not restrictive, and the appended claims are intended to cover all such modifications, enhancements, and other embodiments, which fall within the true scope of the present invention. For example, configurations designated as having superior components and inferior components can be inverted. Thus, to the maximum extent allowed by law, the scope of the present invention is to be determined by the broadest permissible interpretation of the following claims and their equivalents, and shall not be restricted or limited by the foregoing detailed description.

1. A prosthetic device comprising:
a component configured to be implanted in association with two vertebrae, the component comprising a rigid-rod polymeric material.

2. (canceled)

3. The prosthetic device of claim 2, wherein the first surface has a roughness (Ra) not greater than 100 nm.

4. The prosthetic device of claim 1, wherein the rigid-rod polymeric material is self-reinforced and is absent a filler.

5. The prosthetic device of claim 1, wherein the rigid-rod polymeric material has a specific gravity not greater than 1.3 at room temperature.

6. An implantable device comprising:
a component configured to be implanted in association with two vertebrae, the component comprising a polymeric material including a rigid-rod polymer matrix.

7. The implantable device of claim 6, wherein the component is configured to engage at least one of the two vertebrae and facilitate relative motion between the two vertebrae.

8. (canceled)

9. The implantable device of claim 8, wherein the component comprises a core and a coating overlying the core, the coating comprising the rigid-rod polymer material.

10. The implantable device of claim 9, wherein the component is a nucleus prosthetic.

11. The implantable device of claim 9, wherein the core comprises a polymer.

12. The implantable device of claim 11, wherein the polymer is an elastomeric polymer.

13.-16. (canceled)

17. The implantable device of claim 6, wherein the polymeric material consists essentially of the rigid-rod polymer matrix.

18. The implantable device of claim, wherein the polymeric material is substantially free of a filler.

19. The implantable device of claim, wherein the rigid-rod polymer matrix comprises a phenylene-based homopolymer or copolymer.

20. The implantable device of claim 6, wherein the rigid-rod polymer matrix comprises poly(phenylene benzobisthiazole), poly(phenylene benzobisoxazole), poly(phenylene benzimidazole), poly(phenylene terephthalate), poly(benzimidazole), or any combination thereof.

21. The implantable device of claim 6, wherein the polymeric material comprises a polymer blend.

22. The implantable device of claim, wherein the polymer blend is homogeneous.

23. The implantable device of claim, wherein the polymer blend includes the rigid-rod polymer matrix and a second polymer comprising a polyurethane material, a polyolefin material, a polystyrene, a polyurea, a polyamide, a polyaryletherketone (PAEK) material, a silicone material, a hydrogel material, or any alloy, blend or copolymer thereof.

24.-26. (canceled)

27. The implantable device of claim 6, wherein the polymer material comprises a heterogeneous mixture including the rigid-rod polymer matrix and a filler material dispersed therein.

28. The implantable device of claim, wherein the filler material comprises a ceramic, a metal, a carbon, a polymer, or any combination thereof.

29.-35. (canceled)

36. The implantable device of claim 6, wherein the component comprises one or more surfaces coated with an agent.

37. The implantable device of claim, wherein the agent comprises an osteogenerative agent.

38. The implantable device of claim 6, wherein the polymer material has an ultimate tensile strength at room temperature (23° C.) of not less than about 125 MPa.

39. The implantable device of claim 6, wherein the polymer material has an average tensile modulus at room temperature (23° C.) of not less than about 5.00 GPa.

40.-42. (canceled)

43. The implantable device of claim 6, wherein the polymer material has a specific gravity at room temperature of less than about 1.40.

44. (canceled)

45. The implantable device of claim 6, wherein the polymer material comprises substantially isotropic mechanical properties.

46. The implantable device of claim 6, wherein the polymer material has a glass transition temperature of not less than about 145° C.

47. The implantable device of claim 6, wherein the component includes a wear surface comprising the polymeric material.

48. The implantable device of claim, wherein the wear surface has a roughness (Ra) not greater than about 100 nm.

49.-51. (canceled)

52. A prosthetic device comprising:
a first component configured to be implanted in association with two vertebrae, the first component including a first surface configured to moveable engage an opposing second surface, the first surface formed of a rigid-rod polymer; and
a second component including the opposing second surface.

53.-55. (canceled)
